SEQUENCE LISTING (26 sequences)

SEQ ID NO: 1 - DNA, 1209 bp, Homo sapiens
  source 1..1209: mol_type = genomic DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaa

SEQ ID NO: 2 - protein, 21 aa, Thosea asigna virus
GSGEGRGSLLTCGDVEENPGP

SEQ ID NO: 3 - protein, 22 aa, Porcine teschovirus
GSGATNFSLLKQAGDVEENPGP

SEQ ID NO: 4 - protein, 23 aa, Equine rhinitis A virus
GSGQCTNYALLKLAGDVESNPGP

SEQ ID NO: 5 - DNA, 565 bp, Cardiovirus rueckerti
  source 1..565: mol_type = genomic DNA; note = Encephalomyocarditis virus
taacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 6 - DNA, 388 bp, unidentified
  source 1..388: mol_type = unassigned DNA; note = Murine stem cell virus
cgcggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcct

SEQ ID NO: 7 - DNA, 163 bp, Homo sapiens
  source 1..163: mol_type = genomic DNA
agcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaacc

SEQ ID NO: 8 - protein, 172 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 9 - protein, 255 aa, synthetic construct
EQKLISEEDLAGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSITCPPPMSVEHADIWVKSYSLYSRERYICNSGFKRKAGTSSLTECVLNKATNVAHWTTPSLKCIRDGGGGSGGGGSRSNLGWLCLLLLPIPLIVWVKRKEVQKT

SEQ ID NO: 10 - protein, 242 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSITCPPPMSVEHADIWVKSYSLYSRERYICNSGFKRKAGTSSLTECVLNKATNVAHWTTPSLKCIRDGGGGSGGGGSRSNLGWLCLLLLPIPLIVWVKRKEVQKT

SEQ ID NO: 11 - protein, 354 aa, synthetic construct
EQKLISEEDLAGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGGGGGSGGGGSRSNLGWLCLLLLPIPLIVWVKRKEVQKT

SEQ ID NO: 12 - protein, 242 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSITCPPPMSVEHADIWVKSYSLYSRERYICNSGFKRKAGTSSLTECVLNKATNVAHWTTPSLKCIRDGGGGSGGGGSPILLTCPTISILSFFSVALLVILACVLW

SEQ ID NO: 13 - protein, 496 aa, synthetic construct
MLLLVTSLLLCELPHPAFLLIPQVQLVQSGAEVKKPGASVKVSCKTSGYTFTDYYIHWVRQAPGQGLEWMGEIYPGSGNTYYAQKFQGRVTMTADTSTSTAYMELSSLRSEDTAVYYCARGNDYDAWFVYWGQGTLVTVSSGSTSGSGKPGSGEGSTKGDIVMTQSPDSLAVSLGERVTMNCKSSQSLLNSGTRKNYLAWYQQKPGQPPKLLIYWASIRESGVPDRFSGSGSGTDFTLTISSVQAEDVAVYYCKQSYSLYTFGQGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 474 aa, synthetic construct
QVQLVQSGAEVKKPGASVKVSCKTSGYTFTDYYIHWVRQAPGQGLEWMGEIYPGSGNTYYAQKFQGRVTMTADTSTSTAYMELSSLRSEDTAVYYCARGNDYDAWFVYWGQGTLVTVSSGSTSGSGKPGSGEGSTKGDIVMTQSPDSLAVSLGERVTMNCKSSQSLLNSGTRKNYLAWYQQKPGQPPKLLIYWASIRESGVPDRFSGSGSGTDFTLTISSVQAEDVAVYYCKQSYSLYTFGQGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 22 aa, Homo sapiens
MLLLVTSLLLCELPHPAFLLIP

SEQ ID NO: 16 - protein, 119 aa, synthetic construct
QVQLVQSGAEVKKPGASVKVSCKTSGYTFTDYYIHWVRQAPGQGLEWMGEIYPGSGNTYYAQKFQGRVTMTADTSTSTAYMELSSLRSEDTAVYYCARGNDYDAWFVYWGQGTLVTVSS

SEQ ID NO: 17 - protein, 18 aa, synthetic construct
GSTSGSGKPGSGEGSTKG

SEQ ID NO: 18 - protein, 112 aa, synthetic construct
DIVMTQSPDSLAVSLGERVTMNCKSSQSLLNSGTRKNYLAWYQQKPGQPPKLLIYWASIRESGVPDRFSGSGSGTDFTLTISSVQAEDVAVYYCKQSYSLYTFGQGTKLEIK

SEQ ID NO: 19 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 20 - protein, 41 aa, Homo sapiens
KRGRKKLLYIFKQPFMRPVQTTQEEDGCSCRFPEEEEGGCE

SEQ ID NO: 21 - protein, 113 aa, Homo sapiens
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 1488 bp, synthetic construct
  source 1..1488: mol_type = other DNA
atgctgttattagtgacctctttactgctgtgtgagctgccccaccccgctttcctcctcatcccgcaagtccaactggtgcagtccggagccgaggtcaagaagcccggagccagcgtgaaagtctcatgtaaaaccagcggctacaccttcaccgactactacatccactgggtccgacaagcccccggtcaaggtttagagtggatgggcgagatctaccccggctccggcaacacctactacgcccagaagttccaaggtcgtgtgaccatgacagccgacaccagcacctccaccgcctacatggaactgtcctctctgcgttctgaggacacagccgtttactactgcgccagaggcaacgactacgacgcttggttcgtgtactggggccaaggaacattagtgaccgtgtcctccggatccacatccggcagcggaaagcccggtagcggcgagggcagcaccaaaggagacatcgtcatgacccagagccccgattctttagccgtgtctttaggcgaaagagtgaccatgaactgcaagtccagccagtctttactgaattccggcactcgaaaaaactatttagcttggtaccagcagaaacccggccagccccctaagctgctgatctactgggctagcattcgagaatccggcgtgcccgatcgctttagcggcagcggtagcggcaccgactttactttaaccatcagcagcgtgcaagctgaggacgtggctgtgtactattgcaagcagtcctactctttatacaccttcggccaaggaacaaagctggagatcaaggggtccaccacgacgccagcgccgcgaccaccaacaccggcgcccaccatcgcgtcgcaacccctgtccctgcgccccgaggcgtgccggccagcggcggggggcgcagtgcacacgagggggctggacttcgcctgtgatatctacatctgggcgcccttggccgggacttgtggggtccttctcctgtcactggttatcaccctttattgcaaacggggcagaaagaaactcctgtatatattcaaacaaccatttatgagaccagtacaaactactcaagaggaagatggctgtagctgccgatttccagaagaagaagaaggaggatgtgaattgagagtgaagttcagcaggagcgcagacgcccccgcctatcagcaaggccagaaccagctctataacgagctcaatttagggcgaagagaggagtacgatgttttggacaagaggcgtggccgggaccccgaaatggggggaaagccgagaaggaagaaccctcaggaaggcttgtacaatgaattgcagaaggataagatggcggaggcatacagtgagattgggatgaaaggcgagcgccggaggggcaaggggcacgatggcctttatcagggtctcagtacagccaccaaggacacctacgacgcccttcacatgcaagccctgccccctcgc

SEQ ID NO: 23 - protein, 119 aa, synthetic construct
QVQLVQSGAEVKKPGASVKVSCKASGYTFTDYYIHWVRQAPGQGLEWMGEIYPGSGNTYYAQKFQGRVTMTRDTSTSTVYMELSSLRSEDTAVYYCARGNDYDAWFVYWGQGTLVTVSS

SEQ ID NO: 24 - protein, 119 aa, synthetic construct
QVQLVQSGAEVKKPGASVKVSCKTSGYTFTDYYIHWVRQAPGQGLEWIGEIYPGSGNTYYAQKFQGRATLTADTSTSTAYMEFSSLRSEDTAVYYCARGNDYDAWFVYWGQGTLVTVSS

SEQ ID NO: 25 - protein, 112 aa, synthetic construct
DIVMTQSPDSLAVSLGERATINCKSSQSLLNSGTRKNYLAWYQQKPGQPPKLLIYWASIRESGVPDRFSGSGSGTDFTLTISSLQAEDVAVYYCKQSYSLYTFGQGTKLEIK

SEQ ID NO: 26 - DNA, 639 bp, synthetic construct
  source 1..639: mol_type = other DNA
cgcggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccttcgaagtagatctttgtcgatcctaccatccactcgacacacccgccagcggccgctgccaagcttccgagctctcgaattaattcacagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaacc

(19) United States
(12) Patent Application Publication
(10) Pub. No.: US 2025/0382383 A1
(43) Pub. Date: Dec. 18, 2025

(54) TRICISTRONIC CONSTRUCTS FOR ANTI-GPC3 CAR

(71) Applicant: Kite Pharma, Inc., Santa Monica, CA (US)

(72) Inventors: Saikat Banerjee, Oakland, CA (US); Catherine A. Hartzell, Berkeley, CA (US); Edward H. Liao, Petaluma, CA (US); Emily M. Martinez, San Francisco, CA (US)

(21) Appl. No.: 19/222,704

(22) Filed: May 29, 2025

Related U.S. Application Data
(60) Provisional application No. 63/654,815, filed on May 31, 2024.

(51) Int. Cl. C07K 16/30; A61K 39/00; C07K 14/54; C07K 14/71; C12N 15/86

(52) U.S. Cl. CPC C07K 16/303; C07K 14/5443; C07K 14/71; C12N 15/86; A61K 2039/505; C07K 2317/73; C12N 2740/15043; C12N 2840/203

(57) ABSTRACT

Immune cells engineered to express a chimeric antigen receptor (CAR) along with a TGF-beta dominant negative receptor (TGFβ DNR) and/or a membrane-bound IL15 protein (mbIL 15) are provided which are suitable for the treatment of diseases such as cancer.

### CROSS-REFERENCE TO RELATED APPLICATIONS

This application claims the benefit of U.S. Provisional Application No. 63/654,815 filed on May 31, 2024. The entire contents of this application are incorporated herein by reference in its entirety.

### SEQUENCE LISTING

The instant application contains a Sequence Listing, which has been submitted electronically in XML file format, and is hereby incorporated by reference in its entirety. Said XML copy, created on Apr. 23, 2025, is named K-1164-WO-PCT_SL.xml and is 31,067 bytes in size.

### BACKGROUND

Current T cell therapies use enriched or modified human T cells to target and kill cancer cells in a patient. To increase the ability of T cells and NK cells to target and kill a particular cancer cell, methods have been developed to engineer T cells to express constructs which direct T cells or NK cells to a particular target cancer cell. Chimeric antigen receptors (CARs) and engineered T cell receptors (TCRs), which comprise binding domains capable of interacting with a particular tumor antigen, allow T cells to target and kill cancer cells that express the particular tumor antigen.

Glypican 3 (GPC3) is a member of the glypican-related integral membrane heparan sulfate proteoglycans (GRIPS) family that are present on cell surfaces. The protein core of GPC3 consists of two subunits, where the N-terminal subunit has a size of ˜40 kDa and the C-terminal subunit is ˜30 kDa. Six glypicans (GPC1-6) have been identified in mammals. GPC3 plays an important role in modulating the cell proliferation, differentiation, adhesion and migration. GPC3 interacts with both Wnt and frizzled (FZD) to form a complex and triggers downstream signaling. The core protein of GPC3 may serve as a co-receptor or a receiver for Wnt.

GPC3 protein expression is found in HCC, not in normal liver and cholangiocarcinoma. GPC3 is also expressed in melanoma, ovarian clear-cell carcinomas, yolk sac tumors, neuroblastoma, hepatoblastoma, Wilms' tumor cells, and other tumors.

CAR-T cells have been developed for the treatment of various cancers. Their efficacy, it is contemplated, can be further improved by expressing another therapeutic protein, such a cytokine, in the T cells. The expression of multiple exogenous proteins in a cell may be achieved by introducing to the cell one or more expression vectors. A bicistronic or multi-cistronic vector includes multiple coding sequences transcribed into a single mRNA, which can potentially allow expression of multiple proteins.

A major challenge for bicistronic and multi-cistronic vectors, however, is that the translation of the different proteins from the common mRNA may have drastically different efficiency, leading to highly variable expression levels of the various encoded proteins, thereby negatively impacting any therapeutic benefit.

### SUMMARY

Immune cells engineered to express a chimeric antigen receptor (CAR) along with a TGF-beta dominant negative receptor (TGFβ DNR) and/or a membrane-bound IL15 protein (mbIL15), as well as tricistronic and bicistronic constructs encoding these proteins, are provided. These engineered immune cells are suitable for the treatment of diseases such as cancer.

In one embodiment, the present disclosure provides a polynucleotide, comprising a promoter operatively linked to, (a) a first coding sequence encoding a chimeric antigen receptor (CAR), (b) a second coding sequence encoding a TGF-beta dominant negative receptor (TGFβ DNR), (c) a third coding sequence encoding a membrane-bound IL15 protein (mbIL15), (L1) a first linker, between (a) and (b), encoding a first self-cleaving peptide, and (L2) a second linker, between (b) and (c), encoding a second self-cleaving peptide or comprising a ribosome entry site, wherein the CAR comprises an antigen-binding fragment specific to Glypican 3 (GPC3).

In some embodiments, the promoter is an EFla promoter. In some embodiments, the EFla promoter comprises the nucleotide sequence of SEQ ID NO:1.

In some embodiments, the first self-cleaving peptide is a 2A self-cleaving peptide. In some embodiments, the 2A self-cleaving peptide is selected from the group consisting of T2A, P2A, and E2A. In some embodiments, the first self-cleaving peptide comprises T2A. In some embodiments, the T2A comprises the amino acid sequence of SEQ ID NO:2.

In some embodiments, the second linker (L2) comprises the ribosome entry site. In some embodiments, the ribosome entry site is an internal ribosome entry site (IRES). In some embodiments, the IRES is selected from the group consisting of Encephalomyocarditis virus (EMCV) IRES, murine Stem Cell Virus (mSCV) IRES, Picornavirus IRES, Aphthovirus IRES, Kaposi's sarcoma-associated herpesvirus IRES, Hepatitis A IRES, Hepatitis C IRES, Pestivirus IRES, Cripavirus IRES, Rhopalosiphum padi virus IRES, and combinations thereof.

In some embodiments, the IRES is an EMCV IRES. In some embodiments, the EMCV IRES comprises the nucleotide sequence of SEQ ID NO:5.

In some embodiments, the IRES is an mSCV IRES. In some embodiments, the mSCV IRES comprise the nucleotide sequence of SEQ ID NO:6. In some embodiments, the mSCV IRES is followed by a translational enhancer. In some embodiments, the translational enhancer is a SP163 translational enhancer. In some embodiments, the SP163 translational enhancer comprises the nucleotide sequence of SEQ ID NO:7. In some embodiments, the second linker (L2) comprises the nucleotide sequence of SEQ ID NO:26.

In some embodiments, the second linker (L2) encodes a 2A self-cleaving peptide. In some embodiments, the second linker (L2) encodes P2A. In some embodiments, the P2A comprises the amino acid sequence of SEQ ID NO:3.

In some embodiments, the antigen-binding fragment is a single chain fragment (scFv). In some embodiments, the scFv comprises a heavy chain variable region (VH) comprising an amino acid sequence selected from the group consisting of SEQ ID NO:16 and 23-24 and a light chain variable region (VL) comprising an amino acid sequence selected from the group consisting of SEQ ID NO:18 and 25. In some embodiments, the VH comprises the amino acid sequence of SEQ ID NO: 16 and the VL comprises the amino acid sequence of SEQ ID NO: 18.

In some embodiments, the CAR further comprises a CD3 zeta signaling domain. In some embodiments, the CD3 zeta signaling domain comprises the amino acid sequence of SEQ ID NO: 21.

In some embodiments, the CAR comprises the amino acid sequence of SEQ ID NO:14.

In some embodiments, the TGFβ DNR comprises an extracellular domain (ECD) from a TGF-β receptor, and a transmembrane domain (TMD), and lacks amino acid residues responsible for signaling and phosphorylation present in a wild-type TGF-β receptor. In some embodiments, the ECD is from TGF-βRI or TGF-βRII. In some embodiments, the TGFβ DNR comprises the amino acid sequence of SEQ ID NO:8.

In some embodiments, the mbIL 15 comprises an IL-15 domain, a first linker linking the IL-15 domain to an IL-15Rα sushi domain, and a transmembrane domain. In some embodiments, the mbIL15 comprises an amino acid sequence selected from the group consisting of SEQ ID NO:9-12.

In some embodiments, the (a), (b) and (c) in the polynucleotide are disposed sequentially, from proximal to distal, from the promoter.

Also provided, in another embodiment, is a vector comprising the polynucleotide of the present disclosure. In some embodiments, the vector is a plasmid or a viral vector.

Also provided, in some embodiments, is an isolated cell comprising the polynucleotide or the vector of the present disclosure. In some embodiments, the cell expresses the CAR, the TGFβ DNR and the mbIL 15 at a molar ratio of 1:(0.5-2): (0.5-2).

Also provided, in yet another embodiment, is an isolated cell comprising one or more exogenous polynucleotides encoding (a) a chimeric antigen receptor (CAR), (b) a TGF-beta dominant negative receptor (TGFβ DNR), and (c) a membrane-bound IL15 protein (mbIL15), wherein the CAR comprises an antigen-binding fragment specific to Glypican 3 (GPC3), and wherein the CAR, the TGFβ DNR and the mbIL 15 are expressed at molar ratios of 1:(0.5-2): (0.5-2). In some embodiments, the CAR, the TGF-β DNR and the mbIL 15 are expressed at molar ratios of 1:(0.7-1.5):(0.7-1.5).

In some embodiments, the cell is an immune cell. In some embodiments, the immune cell is selected from the group consisting of T cell, NK cell, NKT cell, and macrophage.

Yet further provided is a method or use for treating a cancer in a patient in a need thereof, comprising administering to the patient the cell of the present disclosure. In some embodiments, the patient comprises a cancer cell that expresses GPC3.

In some embodiments, the cancer is selected from the group consisting of hepatocellular carcinoma (HCC), lung squamous cell carcinoma, ovarian carcinoma, gastric carcinoma, melanoma, hepatoblastoma, nephroblastoma, Wilms tumor and a pediatric embryonal tumor. In some embodiments, the cancer is hepatocellular carcinoma (HCC).

### BRIEF DESCRIPTION OF THE FIGURES

FIG. 1 illustrates the structures of the tricistronic and bicistronic constructs used in the studies.

FIG. 2 shows the vector copy numbers (VCN) of the test constructs in transduced T cells.

FIG. 3 shows the cell surface expression levels of the respective proteins from the test constructs in transduced T cells.

FIG. 4 shows the cytotoxicity and cytokine secretion levels of T cells transduced with the test constructs against various negative or antigen expressing target cancer cells.

FIG. 5 presents shows the persistence of CAR-T constructs over time in the absence of exogenous cytokines in the cytokine independent growth assay.

FIG. 6 shows the bulk inhibition of TGF-β induced pSMAD signaling in CAR-T constructs that express the truncated TGF-β DNR.

FIG. 7 shows the mean body weight changes of mice administered the T cells transduced with the test constructs.

FIG. 8A-8B show the tumor burden in the treated animals; A: mean tumor volumes, B: individual tumor volumes.

FIG. 9A-9B show the peripheral CAR+ T-cell expansion levels; A: mean expansion levels, B: individual expansion levels.

FIG. 10 shows the specific inhibition of TGF-β induced pSMAD signaling on CAR-T positive constructs expressing the truncated TGF-β DNR in the phosphoflow SMAD phosphorylation assay.

FIG. 11 shows the mean body weight changes of mice administered the T cells transduced with the test constructs in the tumor rechallenge model.

FIG. 12A-12B show the tumor burden and peripheral CAR+ T-cell expansion levels in the treated animals in the tumor rechallenge model; A: individual tumor volumes of primary tumor (right flank) and tumor from rechallenge on opposite left flank, B: individual peripheral expansion CAR+ levels.

FIG. 13 shows the mean body weight changes of mice administered the T cells transduced with the test constructs manufactured and harvested at D3, D5 and D8.

FIG. 14A-14B show the tumor burden and peripheral CAR+ T-cell expansion levels in the treated animals for CAR+ T-cells manufactured and harvested at D3, D5 and D8; A: individual tumor volumes, B: individual peripheral CAR+ expansion levels.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

It is to be further understood that wherever aspects are described herein with the language “comprising,” otherwise analogous aspects described in terms of “consisting of” and/or “consisting essentially of” are also provided.

Additionally, the terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (i.e., ±10%). For example, about 3 mg can include any number between 2.7 mg and 3.3 mg (for 10%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acctylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that includes at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F (ab′)₂, F (ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the Vu with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ₁-γ₄). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule includes two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, cpitopc-binding fragments, e.g., Fab, Fab′ and F(ab′)₂, Fd, Fvs, single-chain Fvs (scFv), single-chain antibodies, disulfide-linked Fvs (sdFv), fragments including either a VK or V_{H} domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Tricistronic Constructs

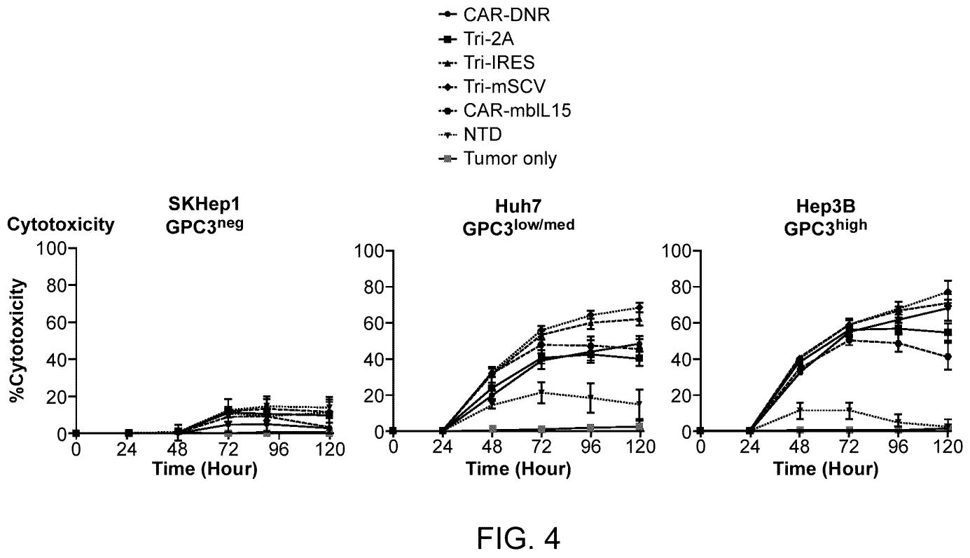

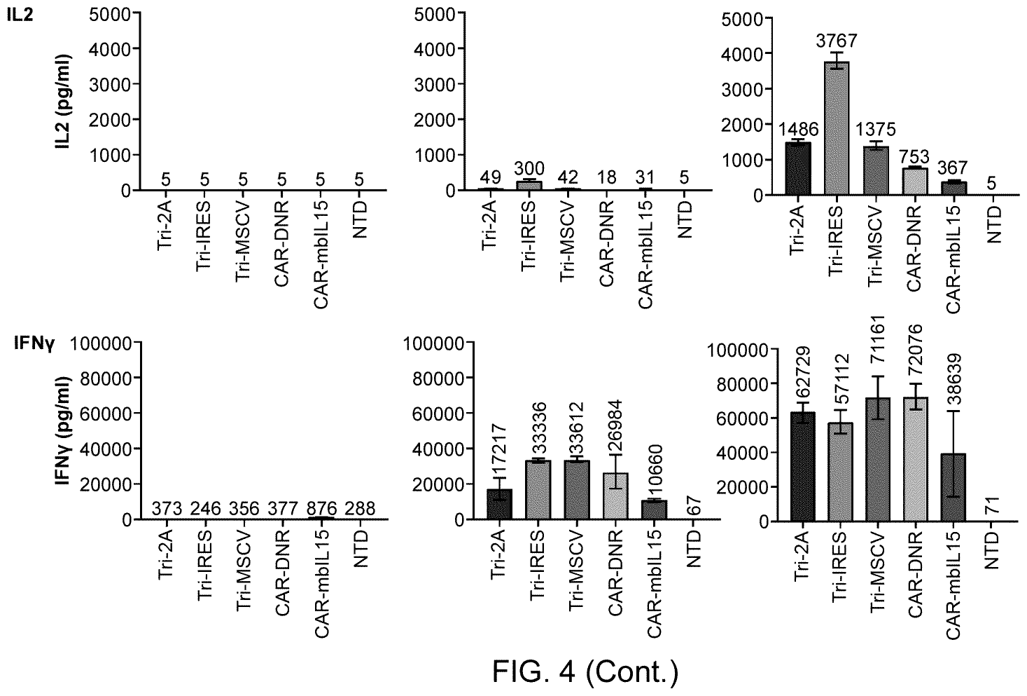

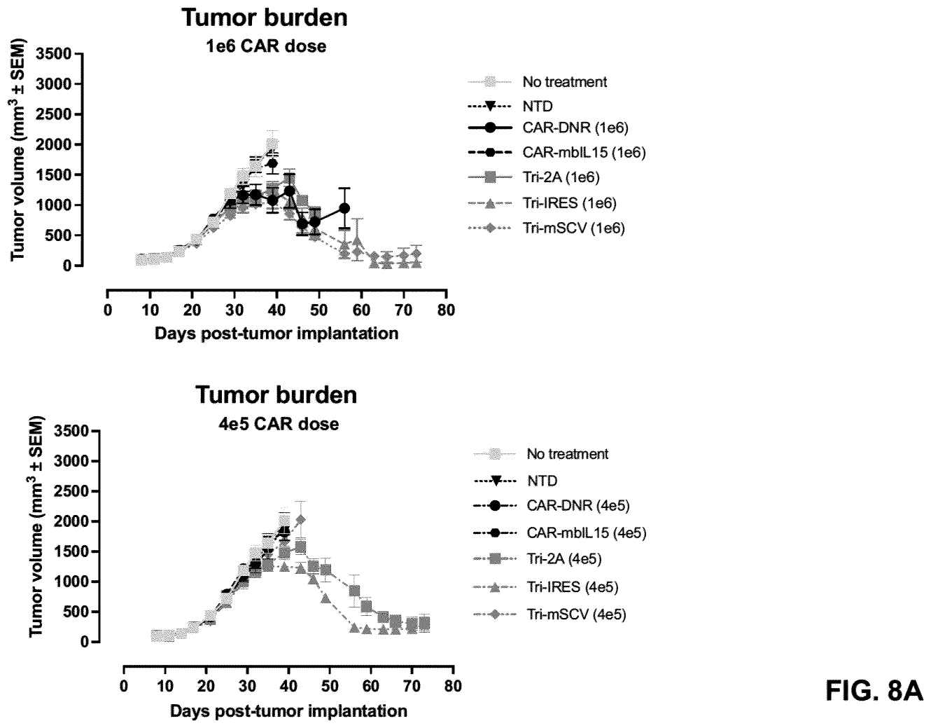

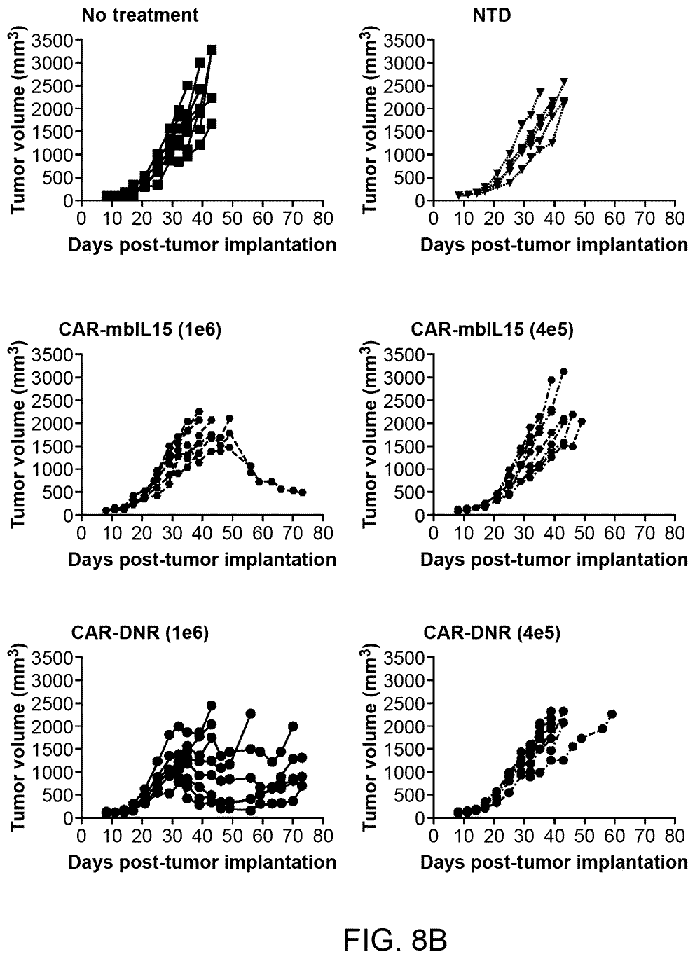

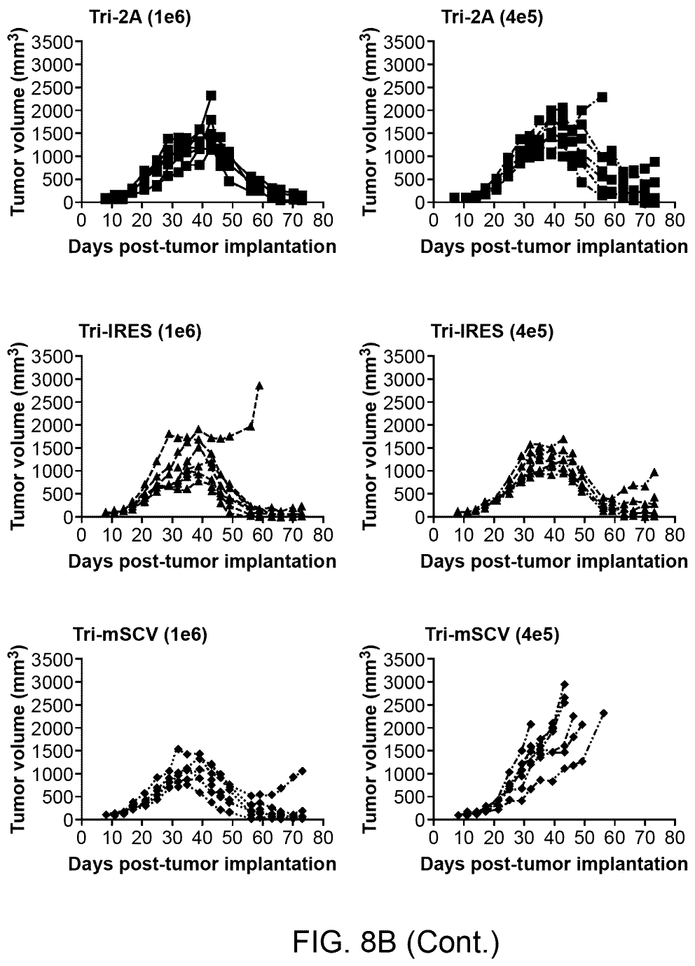

The accompanying experimental examples tested the in vitro and in vivo anti-tumor activities of GPC3 CAR-T cells that further expressed a TGF-beta dominant negative receptor (TGFβ DNR) and/or a membrane-bound IL15 protein (mbIL15). As shown in FIG. 4 (in vitro cytotoxicity) and FIG. 8A-8B (in vivo anti-tumor efficacy), T cells with all three proteins expressed had markedly higher anti-tumor activities than those that expressed the GPC3 CAR along with just one of TGFβ DNR or mbIL15.

Most strikingly, as shown in FIG. 8B, at a 1×10⁶ (1c6) dose, all but two animals treated with T cells expressing GPC3 CAR+mbIL15 (“CAR-mbIL15”) died within 50 days; also, most animals treated with T cells expressing GPC3 CAR+ TGFβ DNR (“CAR-DNR”) cither died by day 50 or had tumors that started to re-grow. By contrast, the vast majority of animals treated with T cells expressing all three proteins were close to tumor-free on the last day of the study (Day 73). This contrast, therefore, underscores the superior anti-tumor efficacy of the GPC3 CAR-T cells enhanced with both TGFβ DNR and mbIL15 expression.

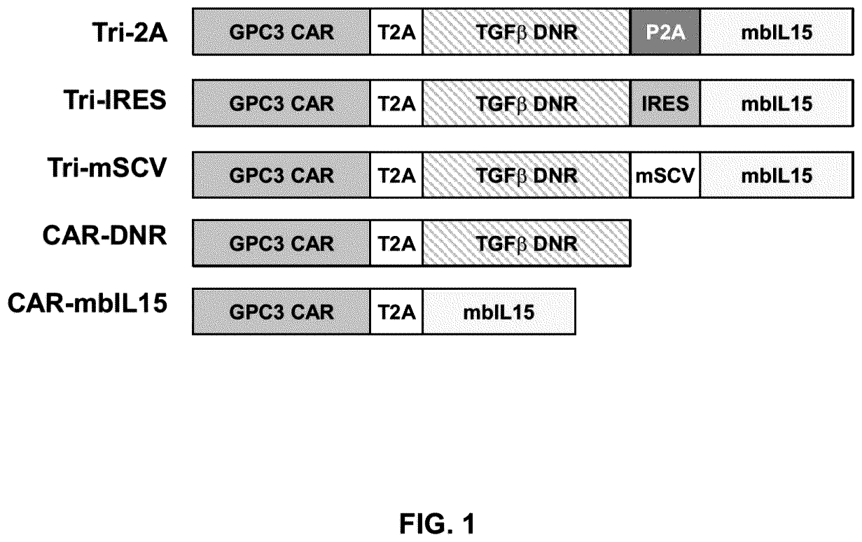

In the experimental examples, when all three surface-bound proteins were expressed, the T cells were transduced with a tricistronic construct that included coding sequences for all three proteins (tricistronic GPC3 CAR-T); when only a TGFβ DNR or a mbIL 15 was added, the T cells were transduced with a bicistronic construct (bicistronic GPC3 CAR-T). The structures of these tricistronic and bicistronic constructs are illustrated in FIG. 1.

In all three tricistronic constructs of the experimental examples (“Tri-2A”, “Tri-IRES”, and “Tri-mSCV”, see FIG. 1), the coding sequences for GPC3 CAR, TGFβ DNR and mbIL15 were arranged sequentially from 5′ to 3′. Also, in all three tricistronic constructs of the experimental examples, a T2A self-cleaving peptide separates the GPC3 CAR and the TGFβ DNR. Uniquely, in Tri-2A, another 2A self-cleaving peptide (P2A) is disposed between the TGFβ DNR and the mbIL15; in Tri-IRES, an Encephalomyocarditis virus (EMCV) internal ribosome entry site (IRES) is used instead; and in Tri-mSCV, a murine Stem Cell Virus (mSCV) IRES is used, followed by a SP163 translational enhancer derived from the 5′UTR of the murine VEGF gene.

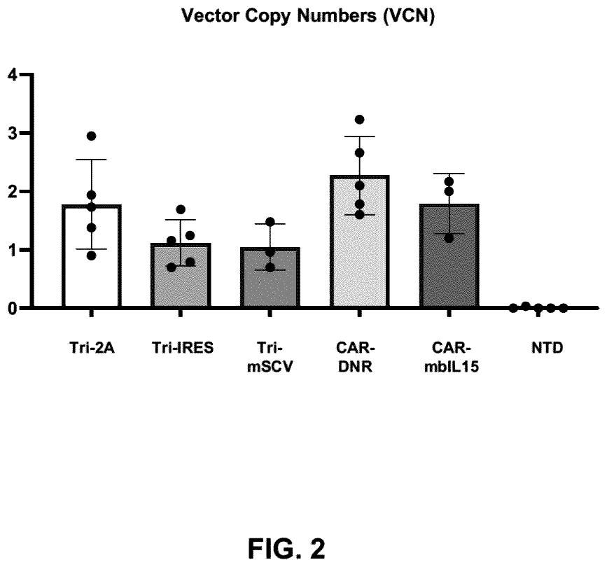

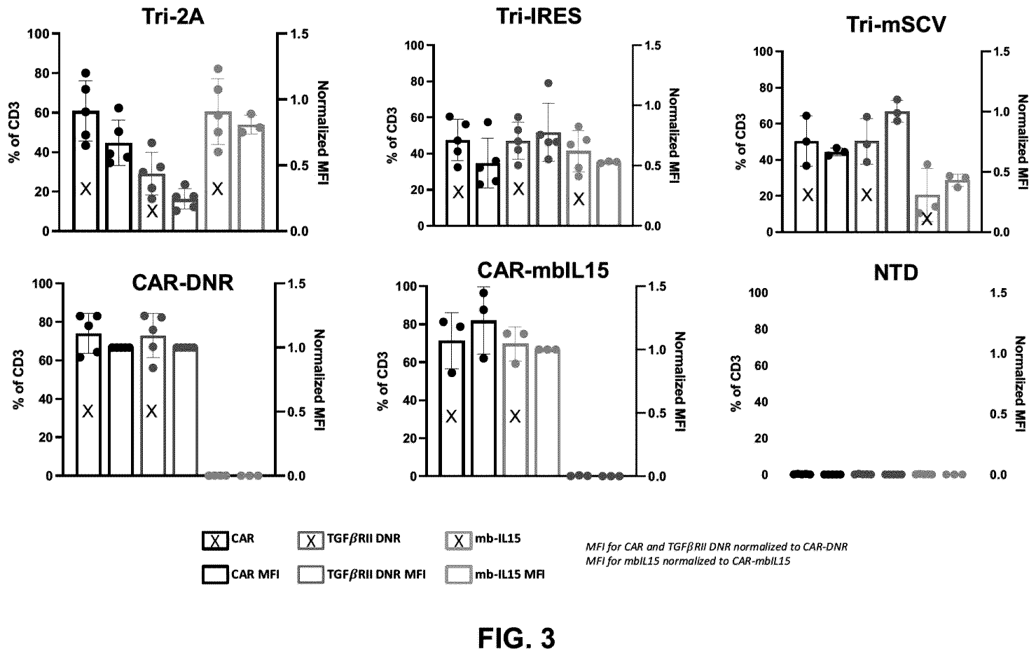

Despite carrying markedly longer coding sequences than the counterpart bicistronic constructs, the tricistronic ones exhibited similar vector copy numbers (VCN) and cell-surface expression levels (FIG. 2-3). Tri-IRES was most able to express the three different proteins at similar levels (FIG. 3).

In accordance with one embodiment of the present disclosure, therefore, provided is a polynucleotide that includes (a) a first coding sequence encoding a chimeric antigen receptor (CAR), (b) a second coding sequence encoding a TGF-beta dominant negative receptor (TGFβ DNR), and (c) a third coding sequence encoding a membrane-bound IL15 protein (mbIL15).

In some embodiments, the CAR has specificity to GPC3. In some embodiments, the polynucleotide further includes a promoter operatively linked to one or more of the foregoing coding sequences. In some embodiments, the promoter is an EFla promoter, such as SEQ ID NO: 1 (see sequences in Table A).

In some embodiments, the coding sequences are arranged, from 5′ to 3′ on the sense strand, in a (a)-(b)-(c) orientation. In some embodiments, the coding sequences are arranged, from 5′ to 3′ on the sense strand, in a (c)-(b)-(a) orientation. In some embodiments, the coding sequences are arranged, from 5′ to 3′ on the sense strand, in a (a)-(c)-(b) orientation. In some embodiments, the coding sequences are arranged, from 5′ to 3′ on the sense strand, in a (b)-(c)-(a) orientation. In some embodiments, the coding sequences are arranged, from 5′ to 3′ on the sense strand, in a (b)-(a)-(c) orientation. In some embodiments, the coding sequences are arranged, from 5′ to 3′ on the sense strand, in a (c)-(a)-(b) orientation.

In some embodiments, at least one of the coding sequences is provided in a separate polynucleotide/vector, or under the regulation of a different promoter within the same polynucleotide/vector as the other two proteins. Such a configuration, with two separate transcription cassettes will then result in the transcription of two mRNA transcripts. In one embodiment, the two transcription cassettes include (a)-(b) and (c), respectively. In onc embodiment, the two transcription cassettes include (b)-(a) and (c), respectively. In one embodiment, the two transcription cassettes include (c)-(b) and (a), respectively. In one embodiment, the two transcription cassettes include (a)-(c) and (b), respectively. In one embodiment, the two transcription cassettes include (b)-(c) and (a), respectively. In one embodiment, the two transcription cassettes include (c)-(a) and (b), respectively. In some embodiments, each of the (a), (b) and (c) are disposed within separate transcription cassettes.

In some embodiments, the coding sequences are arranged, from 5′ to 3′ on the sense strand, in a (a)-(b)-(c) orientation, and a first linker (L1) is disposed between (a) and (b). In some embodiments, a second linker (L2) is disposed between (b) and (c).

In some embodiments, the first linker (L1) encodes a first self-cleaving peptide. An example self-cleaving peptide is a 2A self-cleaving peptide. Non-limiting examples of 2A self-cleaving peptides include T2A (SEQ ID NO:2), P2A (SEQ ID NO:3), and E2A (SEQ ID NO:4). In a particular embodiment, the first self-cleaving peptide is T2A.

In some embodiments, the second linker (L2) encodes a second self-cleaving peptide or includes a ribosome entry site. The second self-cleaving peptide can also be a 2A self-cleaving peptide. Non-limiting examples of 2A self-cleaving peptides include T2A (SEQ ID NO:2), P2A (SEQ ID NO:3), and E2A (SEQ ID NO:4). In a particular embodiment, the second self-cleaving peptide is P2A.

The second linker (L2), in some embodiments, includes a ribosome entry site, such as an internal ribosome entry site (IRES). IRES is an RNA element that allows for translation initiation in a cap-independent manner. IRES elements, therefore, allow ribosomes to engage the mRNA and begin translation of a coding sequence in a multi-cistronic construct.

Examples of IRES sequences include, without limitation, Encephalomyocarditis virus (EMCV) IRES, murine Stem Cell Virus (mSCV) IRES, Picornavirus IRES, Aphthovirus IRES, Kaposi's sarcoma-associated herpesvirus IRES, Hepatitis A IRES, Hepatitis C IRES, Pestivirus IRES, Cripavirus IRES, Rhopalosiphum padi virus IRES, and combinations thereof.

In one embodiment, the IRES is an EMCV IRES (SEQ ID NO:5). In another embodiment, the IRES is an mSCV IRES (SEQ ID NO:6).

In some embodiments, the IRES is followed by a translational enhancer. In some embodiments, the translational enhancer follows the mSCV IRES. In some embodiments, the translational enhancer is a SP163 translational enhancer (SEQ ID NO:7). In some embodiments, the second linker (L2) that includes both the mSCV IRES and the SP163 translational enhancer has the nucleic acid sequence of SEQ ID NO:26.

In some embodiments, the polynucleotide is provided in a vector. The vector, in some embodiments, is a plasmid or a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors.

Various examples of the CAR, TGFβ DNR and mbIL15 suitable for inclusion in the above embodiments are described below in further details.

### A. Chimeric Antigen Receptors (CAR)

Chimeric antigen receptors (CAR) can be engineered to bind to an antigen (such as a cell-surface antigen) by incorporating an antigen binding molecule that interacts with that targeted antigen. Preferably, the antigen binding molecule is an antibody fragment thereof, and more preferably one or more single chain antibody fragment (“scFv”). An scFv is a single chain antibody fragment having the variable regions of the heavy and light chains of an antibody linked together. It will be appreciated that the antigen binding molecule is typically contained within the extracellular portion of the CAR such that it is capable of recognizing and binding to the antigen of interest. Bispecific and multispecific CARs are contemplated within the scope of the disclosure, with specificity to more than one target of interest.

An example CAR includes, in addition to the antigen binding molecule, a transmembrane domain and an activation domain. In some embodiments, the CAR can further include an extracellular domain between the antigen binding molecule and the transmembrane domain. The extracellular domain may include a hinge domain. In some embodiments, the CAR can further include a co-stimulatory domain.

The antigen binding portion of the CAR of the instant disclosure can be an antibody or antigen binding fragment (in particular a scFv) targeting the human GPC3 protein. The antibody or antigen-binding fragment includes a heavy chain variable region (V_{H}) and a light chain variable region (V_{L}). In some embodiments, the V_{H} includes the amino acid sequence of SEQ ID NO: 16, 23 or 24. In some embodiments, the V_{L} includes the amino acid sequence of SEQ ID NO: 18 or 25. In some embodiments, the V_{H} includes the amino acid sequence of SEQ ID NO: 16 and the V_{L} includes the amino acid sequence of SEQ ID NO:18. In some embodiments, the antigen-binding fragment is a scFv in which is a peptide linker (e.g., SEQ ID NO:17) between the V_{H} and the V_{L}. In some embodiments, the orientation in the scFv is V_{H}-linker-V_{L}; and in some embodiments, the orientation is V_{L}-linker-V_{H}.

Chimeric antigen receptors can include an extracellular domain. Extracellular domains often include a hinge portion, sometimes referred to as the “spacer” region. A variety of hinges can be employed in accordance with the disclosure, including portions or derivatives of the molecules as listed above. In certain embodiments, the hinge portion is a hinge region of IgG1, IgG2, IgG3, IgG4, IgA, IgD, IgE, IgM, CD28, or CD8 alpha, any truncation thereof, or any combination thereof. In certain embodiments, the hinge region is a hinge region of CD8 alpha.

The CAR can be designed with a transmembrane domain™. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein.

Non-limiting examples of such proteins include CD28, CD8alpha, CD8beta, 4-1BB, B7-H3, BAFFR, BLAME, BTLA, CD100, CD103, CD11a, CD11b, CD11c, CD11d, CD160, CD18, CD19, CD19a, CD2, CD247, CD27, CD276, CD29, CD3 delta, CD3 epsilon, CD3 gamma, CD30, CD4, CD40, CD49a, CD49D, CD49f, CD69, CD7, CD79a, CD84, CD96, CDS, CEACAMI, CRT AM, DAP-10, DNAMI, Fc gamma receptor, GADS, GITR, HVEM, IA4, ICAM-1, ICOS, IL-2R beta, IL-2R gamma, IL-7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly9, NKG2C, NKG2D, NKp30, NKp44, NKp46, NKp80, OX-40, PAG, PD-1, PSGLI, SELPLG, SLAM, SLAMF4, SLAMF6, SLAMF7, SLP-76, TNFR2, TNFSF14, TRANCE, VLA1, VLA-6, a cytokine receptor, a MHC class 1 molecule, a SLAM protein, a TNF receptor protein, a Toll ligand receptor, an activating NK cell receptor, an immunoglobulin protein, and an integrin. In a particular example, the transmembrane domain is one from CD8a.

In some embodiments, both the hinge and the transmembrane domain™ are derived from the human CD8x protein. An example sequence is provided in SEQ ID NO: 19, or one having at least 80%, 85%, 90%, 95%, 98% or 99% sequence identity to SEQ ID NO: 19.

The intracellular (cytoplasmic) domain of the CAR of the disclosure can provide activation of at least one of the normal effector functions of the immune cell. Effector function of a T cell, for example, may refer to cytolytic activity or helper activity, including the secretion of cytokines. The intracellular domain may include at least an activation domain. The intracellular domain can also include one or more costimulatory domains.

A “costimulatory domain” as used herein refers to a molecule that provides a signal which mediates a T cell response, including, but not limited to, proliferation, activation, differentiation, and the like. Costimulatory domain can provide a signal in addition to the primary signal provided by an activating molecule as described herein.

Chimeric antigen receptors may incorporate costimulatory (signaling) domains to increase their potency. For example, CD28 is a costimulatory protein found naturally on T-cells. A variety of costimulatory molecules are set forth herein, but it will be appreciated that additional costimulatory molecules are also included within the scope of this disclosure.

It will be appreciated that suitable costimulatory domains within the scope of the disclosure include the signaling domain (or other suitable portion) of 2B4, 4-1BB, B7-H3, BAFFR, BLAME, BTLA, CD100, CD103, CD11a, CD11b, CD11c, CD11d, CD150, CD160, CD18, CD19, CD19a, CD2, CD247, CD27, CD276, CD28, CD29, CD3 delta, CD3 epsilon, CD3 gamma, CD30, CD4, CD40, CD49a, CD49D, CD49f, CD69, CD7, CD84, CD8alpha, CD8beta, CD96, CDS, CEACAMI, CRT AM, cytokine receptor, DAP-10, DNAMI, Fc gamma receptor, GADS, GITR, HVEM, IA4, ICAM-1, ICOS, Ig alpha, IL-2R beta, IL-2R gamma, IL-7R alpha, integrin, IPO-3, ITGA4, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly 108, Ly9, MHC class 1 molecule, NKG2C, NKG2D, NKp30, NKp44, NKp46, NKp80, OX-40, PAG, PD-1, PSGLI, SELPLG, SLAMF4, SLAMF6, SLAMF7, SLP-76, TNFR2, TNFSF14, TRANCE, VLA1, VLA-6, a TNF receptor protein, a ligand to CD83, a Toll ligand receptor, an activating NK cell receptor, or an immunoglobulin. It will be appreciated that additional costimulatory molecules, or fragments thereof, not listed above are within the scope of the disclosure.

In a particular example, the costimulatory domain is one from 4-1BB, such as SEQ ID NO: 20, or one having at least 80%, 85%, 90%, 95%, 98% or 99% sequence identity to SEQ ID NO: 20.

An “activation domain” refers to a molecule on a T cell, e.g., the TCR/CD3 complex that specifically binds with a cognate stimulatory ligand present on an antigen present cell. Suitable activating molecules are described herein. The activation domain is part of the intracellular (cytoplasmic) portion of a CAR. The intracellular domain of a CAR can provide activation of at least one of the normal effector functions of the immune cell. Effector function of a T cell, for example, may refer to cytolytic activity or helper activity, including the secretion of cytokines.

CD3 is an element of the T cell receptor on native T cells, and has been shown to be an important intracellular activating element in CARs. In a preferred embodiment, the CD3 is CD3 zeta, CD3 epsilon, CD3 delta, or CD3 gamma.

A commonly used activation domain is the CD3zeta domain by itself or combined with any other desired intracellular domain(s). CD3 is an element of the T cell receptor on native T cells, and has been shown to be an important intracellular activating element in CARs. Example sequences of CD3zeta include SEQ ID NO:21 and sequences having at least 80%, 85%, 90%, 95%, 98% or 99% sequence identity to SEQ ID NO:21.

In some embodiments, the CAR further includes a signal peptide, such as SEQ ID NO: 15. An example GPC3 CAR amino acid sequence is provided in SEQ ID NO:13. A corresponding nucleic acid sequence encoding the GPC3 CAR is SEQ ID NO:22. Upon cleavage of the signal peptide, the cell-surface CAR has the sequence of SEQ ID NO:14.

More examples of GPC3 CAR are described in PCT publication No. WO2022261061A1, the content of which is incorporated into the instant application by reference in its entirety.

B. TGF-beta dominant negative receptor (TGFβ DNR)

Dominant negative TGF-β receptors (TGFβ DNR) are capable of inhibiting the immunosuppressive effects of TGF-β in the tumor microenvironment (TME). These receptors may also stimulate cytokine signaling to enhance T cell function in the TME. A TGFβ DNR may be derived from a wild-type TGF-β Receptor Type I (TGF-βRI) or TGF-β Receptor Type II (TGF-βRII).

Wild-type TGF-β Receptor Type I (TGF-βRI) is the portion of the TGF-β receptor complex that transmits the intracellular, suppressive pSMAD signaling scheme. The full length wild-type sequence for TGF-βRI is provided in NP_004603.1. It includes a signal peptide (at approximately amino acids 1-33), an extracellular domain (at approximately amino acids 34 126), a transmembrane domain (at approximately amino acids 127-147) and an intracellular domain (at approximately amino acids 148-503). The intracellular domain includes four, key threonine sites located between amino acids 185-204 of the intracellular domain of TGF-βRI and initiates pSMAD signaling.

In one embodiment, a dominant negative TGF-βRI can inhibit the phosphorylation cascade and thus limit the immunosuppressive effects of TGF-β on T cell function. Any truncation or modification that results in suppressing or reducing phosphorylation such that it results in a non-functional signaling pathway is within the scope of the disclosure. In one embodiment the TGF-βRI is truncated after the transmembrane domain or modified, such that the polypeptide lacks one or more amino acid residues responsible for signaling and phosphorylation and results in a non-functional signaling pathway as compared to the wild-type receptor. In some embodiments, the TGF-βRI may be truncated before the threonine at amino acid 185 of the intracellular domain. In some embodiments, the TGF-βRI may be truncated such that it has no intracellular domain. In other embodiments, the TGF-βRI may be modified to replace the intracellular domain with another natural or non-naturally occurring sequence that does not include amino acid residues involved in phosphorylation signaling. In another embodiment, the TGF-βRI is truncated or modified in such a way so as to reduce phosphorylation activity. In another embodiment, the TGF-βRI is truncated or modified in such a way so as to inhibit phosphorylation signaling molecules from interacting with pSMAD molecules. In some embodiments, the TGF-βRI is engineered to remove the amino acids involved in phosphorylation signaling of the intracellular domain.

In some embodiments, the extracellular domain of the TGF-βRI may be engineered to recognize and bind to the target TGF-β molecule in order to initiate oligomerization of the complex of TGF-βRII with the DN TGF-βRI. Furthermore, the present disclosure contemplates the binding of DN TGF-βRI to all TGF-β isoforms, including transforming growth factor β Type I (TGF-β1), transforming growth factor β Type II (TGF-β2), transforming growth factor β Type III (TGF-β3) and transforming growth factor β Type IV (TGF-β4). In some embodiments, the TGFβ DNR binds TGF-β1, TGF-β2, TGF-β3 and/or TGF-β4. In one embodiment, the TGFβ DNR binds TGF-β1.

In addition to TGF-βRI, TGF-βRII is the second member of the TGF-β receptor complex. Unlike wild-type TGF-βRI, wild-type TGF β Receptor Type II (TGF-βRII) is constitutively active. Upon binding of the TGF-β ligand and formation of the TGF-βRI dimer-/TGF-βRII dimer-complex, the cytoplasmic domain of TGF-βRII phosphorylates TGF-βRI.

Thus, TGF-βRII is responsible for activating TGF BRI and initiating the subsequent intracellular signal transduction cascade that results in pSMAD signaling. The full length wild-type sequence for TGF-βRII is provided in NP_003233.4. TGF-βRII includes a signal peptide (at approximately amino acids 1-22), an extracellular domain (at approximately amino acids 33-170), a transmembrane domain (at approximately amino acids 171-201) and an intracellular domain (at approximately amino acids 202-567).

In some embodiments, a TGFβ DNR derived from TGF-βRII is unable to initiate phosphorylation of TGF-βRI by omitting the amino acid sequences responsible for signal initiation of phosphorylation, and thus also suppress pSMAD signal transduction. Any truncation or modification that results in suppressing or reducing phosphorylation such that it results in a non-functional signaling pathway is contemplated by the disclosure. In one embodiment the TGF-βRII may be truncated after the transmembrane domain or modified, such that the polypeptide lacks one or more amino acid residues responsible for signaling and phosphorylation and results in a non-functional signaling pathway as compared to the wild-type receptor. In some embodiments, the TGF-βRII may be truncated such that it has no intracellular domain. In other embodiments, the TGF-βRII may be modified to replace the intracellular domain with another natural or non-naturally occurring sequence that does not include amino acid residues involved in phosphorylation signaling. In another embodiment, the polypeptide is truncated or modified in such a way so as to reduce phosphorylation activity. In another embodiment, the polypeptide is truncated or modified in such a way so as to inhibit phosphorylation signaling molecules from interacting with pSMAD molecules. Thus, in one embodiment described herein is a TGFβ DNR derived from TGF-βRII, where the TGF-βRII is truncated after the transmembrane domain.

An example sequence of a TGFβ DNR is provided SEQ ID NO:8, or is a sequence having 80%, 85%, 90%, 95%, 98% or 99% sequence identity to SEQ ID NO:8. In some embodiments, the TGFβ DNR consists essentially of, or consists of SEQ ID NO:8, or a sequence having 80%, 85%, 90%, 95%, 98% or 99% sequence identity to SEQ ID NO:8.

In some embodiments, the TGFβ DNR further includes a signal peptide. More examples of TGFβ DNR are described in PCT publication No. WO2020257823A2, the content of which is incorporated into the instant application by reference in its entirety.

C. Membrane-bound IL15 protein (mbIL15)

Certain embodiments provide chimeric membrane-bound IL15 protein (mbIL15) that can be anchored to the cell membrane. In some embodiments, the mbIL 15 includes a signal peptide which would be cleaved off upon transport to the cell surface. In some embodiments, the mbIL15 includes a transmembrane domain as illustrated below.

Interleukin 15 (IL-15) is a is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). The number of CD8+ memory cells is shown to be controlled by a balance between this cytokine and IL-2. IL-15 induces the activation of JAK kinases, as well as the phosphorylation and activation of transcription activators STAT3, STAT5, and STAT6. The wild-type IL-15 protein can have an amino acid sequence as shown in NCBI Gene ID: 3600.

Interleukin 15 receptor subunit alpha (also known as CD125 or IL-15Rx) is a cytokine receptor that specifically binds interleukin 15 (IL-15) with high affinity. The receptors of IL-15 and IL-2 share two subunits, IL-2R beta and IL-2R gamma. IL-15Ra is structurally related to IL-2R alpha, an additional IL-2-specific alpha subunit for high affinity IL-2 binding. The protein IL-15Ra can have any amino acid sequence known in the art, for example as available in the NCBI Gene ID: 3601.

In some embodiments, the mbIL 15 is an IL-15-IL-15Rα sushi domain chimeric polypeptide. In some embodiments, the membrane-bound IL-15-IL-15Ra sushi domain chimeric polypeptide includes a FAS transmembrane domain sequence. It is contemplated that this sequence results in the surface expression of a monomer membrane-bound IL-15-IL-15Ra sushi domain chimeric polypeptide.

In embodiments, the membrane-bound IL-15-IL-15Rα sushi domain chimeric polypeptide includes a heterologous dimerization domain such that when expressed the membrane-bound IL-15-IL-15Rα sushi domain chimeric polypeptide forms a homodimer.

Non-limiting examples of the mbIL15 are provided in SEQ ID NO:9-12 (after removal of the signal peptide), as well as their analogs such as those having 80%, 85%, 90%, 95%, 98% or 99% sequence identity to any one of SEQ ID NO:9-12. More examples of mbIL15 are described in PCT publication No. WO2022192439A1, the content of which is incorporated into the instant application by reference in its entirety.

TABLE A
Sequences

| | | SEQ |
|---|---|---|
| | | ID |
| Name | Sequence | NO: |
| EF1a | CGTGAGGCTCCGGTGCCCGTCAGTGGGCAGAGCGCACAT | 1 |
| | CGCCCACAGTCCCCGAGAAGTTGGGGGGAGGGGTCGGCA | |
| | ATTGAACCGGTGCCTAGAGAAGGTGGCGCGGGGTAAACT | |
| | GGGAAAGTGATGTCGTGTACTGGCTCCGCCTTTTTCCCGA | |
| | GGGTGGGGGAGAACCGTATATAAGTGCAGTAGTCGCCGT | |
| | GAACGTTCTTTTTCGCAACGGGTTTGCCGCCAGAACACAG | |
| | GTAAGTGCCGTGTGTGGTTCCCGCGGGCCTGGCCTCTTTA | |
| | CGGGTTATGGCCCTTGCGTGCCTTGAATTACTTCCACGCC | |
| | CCTGGCTGCAGTACGTGATTCTTGATCCCGAGCTTCGGGT | |
| | TGGAAGTGGGTGGGAGAGTTCGAGGCCTTGCGCTTAAGG | |
| | AGCCCCTTCGCCTCGTGCTTGAGTTGAGGCCTGGCTTGGG | |
| | CGCTGGGGCCGCCGCGTGCGAATCTGGTGGCACCTTCGC | |
| | GCCTGTCTCGCTGCTTTCGATAAGTCTCTAGCCATTTAAA | |
| | ATTTTTGATGACCTGCTGCGACGCTTTTTTTCTGGCAAGA | |
| | TAGTCTTGTAAATGCGGGCCAAGATCTGCACACTGGTATT | |
| | TCGGTTTTTGGGGCCGCGGGCGGCGACGGGGCCCGTGCG | |
| | TCCCAGCGCACATGTTCGGCGAGGCGGGGCCTGCGAGCG | |
| | CGGCCACCGAGAATCGGACGGGGGTAGTCTCAAGCTGGC | |
| | CGGCCTGCTCTGGTGCCTGGCCTCGCGCCGCCGTGTATCG | |
| | CCCCGCCCTGGGCGGCAAGGCTGGCCCGGTCGGCACCAG | |
| | TTGCGTGAGCGGAAAGATGGCCGCTTCCCGGCCCTGCTG | |
| | CAGGGAGCTCAAAATGGAGGACGCGGCGCTCGGGAGAG | |
| | CGGGCGGGTGAGTCACCCACACAAAGGAAAAGGGCCTTT | |
| | CCGTCCTCAGCCGTCGCTTCATGTGACTCCACGGAGTACC | |
| | GGGCGCCGTCCAGGCACCTCGATTAGTTCTCGAGCTTTTG | |
| | GAGTACGTCGTCTTTAGGTTGGGGGGAGGGGTTTTATGCG | |
| | ATGGAGTTTCCCCACACTGAGTGGGTGGAGACTGAAGTT | |
| | AGGCCAGCTTGGCACTTGATGTAATTCTCCTTGGAATTTG | |
| | CCCTTTTTGAGTTTGGATCTTGGTTCATTCTCAAGCCTCAG | |
| | ACAGTGGTTCAAAGTTTTTTTCTTCCATTTCAGGTGTCGTG | |
| | AAAACTACCCCTAAAAGCCAAA | |
| T2A | GSGEGRGSLLTCGDVEENPGP | 2 |
| P2A | GSGATNFSLLKQAGDVEENPGP | 3 |
| E2A | GSGQCTNYALLKLAGDVESNPGP | 4 |
| EMCV IRES | TAACGTTACTGGCCGAAGCCGCTTGGAATAAGGCCGGTG | 5 |
| | TGCGTTTGTCTATATGTTATTTTCCACCATATTGCCGTCTT | |
| | TTGGCAATGTGAGGGCCCGGAAACCTGGCCCTGTCTTCTT | |
| | GACGAGCATTCCTAGGGGTCTTTCCCCTCTCGCCAAAGGA | |
| | ATGCAAGGTCTGTTGAATGTCGTGAAGGAAGCAGTTCCT | |
| | CTGGAAGCTTCTTGAAGACAAACAACGTCTGTAGCGACC | |
| | CTTTGCAGGCAGCGGAACCCCCCACCTGGCGACAGGTGC | |
| | CTCTGCGGCCAAAAGCCACGTGTATAAGATACACCTGCA | |
| | AAGGCGGCACAACCCCAGTGCCACGTTGTGAGTTGGATA | |
| | GTTGTGGAAAGAGTCAAATGGCTCTCCTCAAGCGTATTCA | |
| | ACAAGGGGCTGAAGGATGCCCAGAAGGTACCCCATTGTA | |
| | TGGGATCTGATCTGGGGCCTCGGTGCACATGCTTTACATG | |
| | TGTTTAGTCGAGGTTAAAAAACGTCTAGGCCCCCCGAAC | |
| | CACGGGGACGTGGTTTTCCTTTGAAAAACACGATGATAA | |
| | TATGGCCACAACC | |
| mSCV | CGCGGAATGAAAGACCCCACCTGTAGGTTTGGCAAGCTA | 6 |
| | GCTTAAGTAACGCCATTTTGCAAGGCATGGAAAATACAT | |
| | AACTGAGAATAGAGAAGTTCAGATCAAGGTTAGGAACAG | |
| | AGAGACAGCAGAATATGGGCCAAACAGGATATCTGTGGT | |
| | AAGCAGTTCCTGCCCCGGCTCAGGGCCAAGAACAGATGG | |
| | TCCCCAGATGCGGTCCCGCCCTCAGCAGTTTCTAGAGAAC | |
| | CATCAGATGTTTCCAGGGTGCCCCAAGGACCTGAAATGA | |
| | CCCTGTGCCTTATTTGAACTAACCAATCAGTTCGCTTCTC | |
| | GCTTCTGTTCGCGCGCTTCTGCTCCCCGAGCTCAATAAAA | |
| | GAGCCCACAACCCCTCACTCGGCGCGCCAGTCCT | |
| SP163 | AGCGCAGAGGCTTGGGGCAGCCGAGCGGCAGCCAGGCCC | 7 |
| | CGGCCCGGGCCTCGGTTCCAGAAGGGAGAGGAGCCCGCC | |
| | AAGGCGCGCAAGAGAGCGGGCTGCCTCGCAGTCCGAGCC | |
| | GGAGAGGGAGCGCGAGCCGCGCCGGCCCCGGACGGCCT | |
| | CCGAAACC | |
| TGFβ DNR | TIPPHVQKSVNNDMIVTDNNGAVKFPQLCKFCDVRFSTCDN | 8 |
| | QKSCMSNCSITSICEKPQEVCVAVWRKNDENITLETVCHDP | |
| | KLPYHDFILEDAASPKCIMKEKKKPGETFFMCSCSSDECND | |
| | NIIFSEEYNTSNPDLLLVIFQVTGISLLPPLGVAISVIIIFYCYRV | |
| | NRQ | |
| mbIL15 v1 | EQKLISEEDLAGSNWVNVISDLKKIEDLIQSMHIDATLYTES | 9 |
| | DVHPSCKVTAMKCFLLELQVISLESGDASIHDTVENLIILAN | |
| | NSLSSNGNVTESGCKECEELEEKNIKEFLQSFVHIVQMFINTS | |
| | SGGGSGGGGSGGGGSGGGGSGGGSITCPPPMSVEHADIWV | |
| | KSYSLYSRERYICNSGFKRKAGTSSLTECVLNKATNVAHWT | |
| | TPSLKCIRDGGGGSGGGGSRSNLGWLCLLLLPIPLIVWVKRK | |
| | EVQKT | |
| mbIL15 v2 | NWVNVISDLKKIEDLIQSMHIDATLYTESDVHPSCKVTAMK | 10 |
| | CFLLELQVISLESGDASIHDTVENLIILANNSLSSNGNVTESG | |
| | CKECEELEEKNIKEFLQSFVHIVQMFINTSSGGGSGGGGSGG | |
| | GGSGGGGSGGGSITCPPPMSVEHADIWVKSYSLYSRERYIC | |
| | NSGFKRKAGTSSLTECVLNKATNVAHWTTPSLKCIRDGGGG | |
| | SGGGGSRSNLGWLCLLLLPIPLIVWVKRKEVQKT | |
| mbIL15 v3 | EQKLISEEDLAGSNWVNVISDLKKIEDLIQSMHIDATLYTES | 11 |
| | DVHPSCKVTAMKCFLLELQVISLESGDASIHDTVENLIILAN | |
| | NSLSSNGNVTESGCKECEELEEKNIKEFLQSFVHIVQMFINTS | |
| | SGGGSGGGGSGGGGSGGGGSGGGSITCPPPMSVEHADIWV | |
| | KSYSLYSRERYICNSGFKRKAGTSSLTECVLNKATNVAHWT | |
| | TPSLKCIRDPALVHQRPAPPSTVTTAGVTPQPESLSPSGKEPA | |
| | ASSPSSNNTAATTAAIVPGSQLMPSKSPSTGTTEISSHESSHG | |
| | TPSQTTAKNWELTASASHQPPGGGGGSGGGGSRSNLGWLC | |
| | LLLLPIPLIVWVKRKEVQKT | |
| mbIL15 v4 | NWVNVISDLKKIEDLIQSMHIDATLYTESDVHPSCKVTAMK | 12 |
| | CFLLELQVISLESGDASIHDTVENLIILANNSLSSNGNVTESG | |
| | CKECEELEEKNIKEFLQSFVHIVQMFINTSSGGGSGGGGSGG | |
| | GGSGGGGSGGGSITCPPPMSVEHADIWVKSYSLYSRERYIC | |
| | NSGFKRKAGTSSLTECVLNKATNVAHWTTPSLKCIRDGGGG | |
| | SGGGGSPILLTCPTISILSFFSVALLVILACVLW | |
| GPC3 CAR | MLLLVTSLLLCELPHPAFLLIPQVQLVQSGAEVKKPGASVK | 13 |
| full sequence | VSCKTSGYTFTDYYIHWVRQAPGQGLEWMGEIYPGSGNTY | |
| | YAQKFQGRVTMTADTSTSTAYMELSSLRSEDTAVYYCARG | |
| | NDYDAWFVYWGQGTLVTVSSGSTSGSGKPGSGEGSTKGDI | |
| | VMTQSPDSLAVSLGERVTMNCKSSQSLLNSGTRKNYLAWY | |
| | QQKPGQPPKLLIYWASIRESGVPDRFSGSGSGTDFTLTISSVQ | |
| | AEDVAVYYCKQSYSLYTFGQGTKLEIKGSTTTPAPRPPTPAP | |
| | TIASQPLSLRPEACRPAAGGAVHTRGLDFACDIYIWAPLAGT | |
| | CGVLLLSLVITLYCKRGRKKLLYIFKQPFMRPVQTTQEEDGC | |
| | SCRFPEEEEGGCELRVKFSRSADAPAYQQGQNQLYNELNLG | |
| | RREEYDVLDKRRGRDPEMGGKPRRKNPQEGLYNELQKDK | |
| | MAEAYSEIGMKGERRRGKGHDGLYQGLSTATKDTYDALH | |
| | MQALPPR | |
| GPC3 CAR | QVQLVQSGAEVKKPGASVKVSCKTSGYTFTDYYIHWVRQA | 14 |
| after removal | PGQGLEWMGEIYPGSGNTYYAQKFQGRVTMTADTSTSTAY | |
| of signal | MELSSLRSEDTAVYYCARGNDYDAWFVYWGQGTLVTVSS | |
| peptide | GSTSGSGKPGSGEGSTKGDIVMTQSPDSLAVSLGERVTMNC | |
| | KSSQSLLNSGTRKNYLAWYQQKPGQPPKLLIYWASIRESGV | |
| | PDRFSGSGSGTDFTLTISSVQAEDVAVYYCKQSYSLYTFGQ | |
| | GTKLEIKGSTTTPAPRPPTPAPTIASQPLSLRPEACRPAAGGA | |
| | VHTRGLDFACDIYIWAPLAGTCGVLLLSLVITLYCKRGRKK | |
| | LLYIFKQPFMRPVQTTQEEDGCSCRFPEEEEGGCELRVKFSR | |
| | SADAPAYQQGQNQLYNELNLGRREEYDVLDKRRGRDPEM | |
| | GGKPRRKNPQEGLYNELQKDKMAEAYSEIGMKGERRRGK | |
| | GHDGLYQGLSTATKDTYDALHMQALPPR | |
| Signal | MLLLVTSLLLCELPHPAFLLIP | 15 |
| peptide | | |
| GPC3 VH | QVQLVQSGAEVKKPGASVKVSCKTSGYTFTDYYIHWVRQA | 16 |
| | PGQGLEWMGEIYPGSGNTYYAQKFQGRVTMTADTSTSTAY | |
| | MELSSLRSEDTAVYYCARGNDYDAWFVYWGQGTLVTVSS | |
| Whitlow | GSTSGSGKPGSGEGSTKG | 17 |
| linker | | |
| GPC3 VL | DIVMTQSPDSLAVSLGERVTMNCKSSQSLLNSGTRKNYLA | 18 |
| | WYQQKPGQPPKLLIYWASIRESGVPDRFSGSGSGTDFTLTIS | |
| | SVQAEDVAVYYCKQSYSLYTFGQGTKLEIK | |
| CD8a Hinge | TTTPAPRPPTPAPTIASQPLSLRPEACRPAAGGAVHTRGLDF | 19 |
| and TM | ACDIYIWAPLAGTCGVLLLSLVITLYC | |
| 4-1BB | KRGRKKLLYIFKQPFMRPVQTTQEEDGCSCRFPEEEEGGCE | 20 |
| costim | | |
| CD3z | LRVKFSRSADAPAYQQGQNQLYNELNLGRREEYDVLDKRR | 21 |
| | GRDPEMGGKPRRKNPQEGLYNELQKDKMAEAYSEIGMKG | |
| | ERRRGKGHDGLYQGLSTATKDTYDALHMQALPPR | |
| GPC3 CAR | ATGCTGTTATTAGTGACCTCTTTACTGCTGTGTGAGCTGC | 22 |
| full | CCCACCCCGCTTTCCTCCTCATCCCGCAAGTCCAACTGGT | |
| nucleotide | GCAGTCCGGAGCCGAGGTCAAGAAGCCCGGAGCCAGCGT | |
| sequence | GAAAGTCTCATGTAAAACCAGCGGCTACACCTTCACCGA | |
| | CTACTACATCCACTGGGTCCGACAAGCCCCCGGTCAAGG | |
| | TTTAGAGTGGATGGGCGAGATCTACCCCGGCTCCGGCAA | |
| | CACCTACTACGCCCAGAAGTTCCAAGGTCGTGTGACCAT | |
| | GACAGCCGACACCAGCACCTCCACCGCCTACATGGAACT | |
| | GTCCTCTCTGCGTTCTGAGGACACAGCCGTTTACTACTGC | |
| | GCCAGAGGCAACGACTACGACGCTTGGTTCGTGTACTGG | |
| | GGCCAAGGAACATTAGTGACCGTGTCCTCCGGATCCACA | |
| | TCCGGCAGCGGAAAGCCCGGTAGCGGCGAGGGCAGCACC | |
| | AAAGGAGACATCGTCATGACCCAGAGCCCCGATTCTTTA | |
| | GCCGTGTCTTTAGGCGAAAGAGTGACCATGAACTGCAAG | |
| | TCCAGCCAGTCTTTACTGAATTCCGGCACTCGAAAAAACT | |
| | ATTTAGCTTGGTACCAGCAGAAACCCGGCCAGCCCCCTA | |
| | AGCTGCTGATCTACTGGGCTAGCATTCGAGAATCCGGCGT | |
| | GCCCGATCGCTTTAGCGGCAGCGGTAGCGGCACCGACTT | |
| | TACTTTAACCATCAGCAGCGTGCAAGCTGAGGACGTGGC | |
| | TGTGTACTATTGCAAGCAGTCCTACTCTTTATACACCTTC | |
| | GGCCAAGGAACAAAGCTGGAGATCAAGGGGTCCACCAC | |
| | GACGCCAGCGCCGCGACCACCAACACCGGCGCCCACCAT | |
| | CGCGTCGCAACCCCTGTCCCTGCGCCCCGAGGCGTGCCG | |
| | GCCAGCGGCGGGGGGCGCAGTGCACACGAGGGGGCTGG | |
| | ACTTCGCCTGTGATATCTACATCTGGGCGCCCTTGGCCGG | |
| | GACTTGTGGGGTCCTTCTCCTGTCACTGGTTATCACCCTTT | |
| | ATTGCAAACGGGGCAGAAAGAAACTCCTGTATATATTCA | |
| | AACAACCATTTATGAGACCAGTACAAACTACTCAAGAGG | |
| | AAGATGGCTGTAGCTGCCGATTTCCAGAAGAAGAAGAAG | |
| | GAGGATGTGAATTGAGAGTGAAGTTCAGCAGGAGCGCAG | |
| | ACGCCCCCGCCTATCAGCAAGGCCAGAACCAGCTCTATA | |
| | ACGAGCTCAATTTAGGGCGAAGAGAGGAGTACGATGTTT | |
| | TGGACAAGAGGCGTGGCCGGGACCCCGAAATGGGGGGA | |
| | AAGCCGAGAAGGAAGAACCCTCAGGAAGGCTTGTACAAT | |
| | GAATTGCAGAAGGATAAGATGGCGGAGGCATACAGTGA | |
| | GATTGGGATGAAAGGCGAGCGCCGGAGGGGCAAGGGGC | |
| | ACGATGGCCTTTATCAGGGTCTCAGTACAGCCACCAAGG | |
| | ACACCTACGACGCCCTTCACATGCAAGCCCTGCCCCCTCG | |
| | C | |
| GPC3 VH v2 | QVQLVQSGAEVKKPGASVKVSCKASGYTFTDYYIHWVRQA | 23 |
| | PGQGLEWMGEIYPGSGNTYYAQKFQGRVTMTRDTSTSTVY | |
| | MELSSLRSEDTAVYYCARGNDYDAWFVYWGQGTLVTVSS | |
| GPC3 VH v3 | QVQLVQSGAEVKKPGASVKVSCKTSGYTFTDYYIHWVRQA | 24 |
| | PGQGLEWIGEIYPGSGNTYYAQKFQGRATLTADTSTSTAYM | |
| | EFSSLRSEDTAVYYCARGNDYDAWFVYWGQGTLVTVSS | |
| GPC3 VL v2 | DIVMTQSPDSLAVSLGERATINCKSSQSLLNSGTRKNYLAW | 25 |
| | YQQKPGQPPKLLIYWASIRESGVPDRFSGSGSGTDFTLTISSL | |
| | QAEDVAVYYCKQSYSLYTFGQGTKLEIK | |
| mSCV- | CGCGGAATGAAAGACCCCACCTGTAGGTTTGGCAAGCTA | 26 |
| SP163 | GCTTAAGTAACGCCATTTTGCAAGGCATGGAAAATACAT | |
| | AACTGAGAATAGAGAAGTTCAGATCAAGGTTAGGAACAG | |
| | AGAGACAGCAGAATATGGGCCAAACAGGATATCTGTGGT | |
| | AAGCAGTTCCTGCCCCGGCTCAGGGCCAAGAACAGATGG | |
| | TCCCCAGATGCGGTCCCGCCCTCAGCAGTTTCTAGAGAAC | |
| | CATCAGATGTTTCCAGGGTGCCCCAAGGACCTGAAATGA | |
| | CCCTGTGCCTTATTTGAACTAACCAATCAGTTCGCTTCTC | |
| | GCTTCTGTTCGCGCGCTTCTGCTCCCCGAGCTCAATAAAA | |
| | GAGCCCACAACCCCTCACTCGGCGCGCCAGTCCTTCGAA | |
| | GTAGATCTTTGTCGATCCTACCATCCACTCGACACACCCG | |
| | CCAGCGGCCGCTGCCAAGCTTCCGAGCTCTCGAATTAATT | |
| | CACAGCGCAGAGGCTTGGGGCAGCCGAGCGGCAGCCAG | |
| | GCCCCGGCCCGGGCCTCGGTTCCAGAAGGGAGAGGAGCC | |
| | CGCCAAGGCGCGCAAGAGAGCGGGCTGCCTCGCAGTCCG | |
| | AGCCGGAGAGGGAGCGCGAGCCGCGCCGGCCCCGGACG | |
| | GCCTCCGAAACC | |

CAR-Immune Cells Expressing TGFβDNR and mbIL15

Immune cells expressing/enclosing a CAR of the present disclosure or one or more polynucleotides encoding the CAR are also provided. In some embodiments, the immune cells further enclose or express TGFβ DNR and/or mbIL15.

In some embodiments, the CAR, TGFβ DNR and/or mbIL15 are encoded by a tricistronic or bicistronic construct as described above. In some embodiments, they can be encoded by separate polynucleotides, constructs or vectors.

In one embodiment, the present disclosure provides a cell comprising one or more exogenous polynucleotides encoding (a) a chimeric antigen receptor (CAR), (b) a TGF-beta dominant negative receptor (TGFβ DNR), and (c) a membrane-bound IL 15 protein (mbIL15). In some embodiments, the CAR has specificity to Glypican 3 (GPC3).

In some embodiments, the three proteins (the CAR, the TGFβ DNR and the mbIL15) are expressed at similar molar levels. In one embodiments, the molar ratio of the CAR, the TGFβ DNR and the mbIL 15 is within 1:(0.5-2): (0.5-2). In one embodiments, the molar ratio of the CAR, the TGFβ DNR and the mbIL15 is within 1:(0.6-1.8): (0.6-1.8). In one embodiments, the molar ratio of the CAR, the TGFβ DNR and the mbIL15 is within 1:(0.7-1.5):(0.7-1.5). In one embodiments, the molar ratio of the CAR, the TGFβ DNR and the mbIL15 is within 1:(0.8-1.4): (0.8-1.4). In one embodiments, the molar ratio of the CAR, the TGFβ DNR and the mbIL15 is within 1:(0.9-1.2): (0.9-1.2).

Various types of immune cells have been tested for expressing CARs. Non-limiting examples include T cells, NK cells, macrophages and monocytes. A T cell, in some embodiment, may be an alpha beta T cell. In some embodiments, the T cell is a gamma delta T cell.

NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO-, CCR7+, CD45RA+, CD62L+ (L-selectin), CD27+, CD28+ and IL-7Ra+, but they also express large amounts of CD95, IL-2RB, CXCR3, and LFA-1, and show numerous functional attributes distinctive of memory cells); (ii) central memory Tcm cells express L-selectin and the CCR7, they secrete IL-2, but not IFNγ or IL-4; (iii) effector memory TEM cells, however, do not express L-selectin or CCR7 but produce effector cytokines like IFNγ and IL-4), Regulatory T-cells (Tregs, suppressor T cells, or CD4+CD25+ regulatory T cells and (iv) T_{EMRA} cells, effector memory cells re-expressing CD45RA (“terminally differentiated cells”)), Natural Killer T-cells (NKT) and Gamma Delta T-cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

In some embodiments, the immune cell is autologous, or allogeneic. Without limitation, an autologous immune cell is obtained from a patient and engineered to express a CAR. An allogeneic immune cell may be derived from donor cells, such as stem cells.

Methods of preparing the engineered immune cells are also provided. In some embodiments, the method entails transfecting or transducing an immune cell with a polynucleotide or vector encoding the CAR and the other proteins.

Methods of Treatments with Engineered Immune Cells

Using adoptive immunotherapy, native immune cells (e.g., T cells) can be (i) removed from a patient, (ii) genetically engineered to express a chimeric antigen receptor (CAR) that binds to at least one tumor antigen (iii) expanded ex vivo into a larger population of engineered immune cells, and (iv) reintroduced into the patient. After the engineered immune cells are reintroduced into the patient, they mediate an immune response against cells expressing the tumor antigen. This immune response includes secretion of IL-2 and other cytokines by T cells, the clonal expansion of T cells recognizing the tumor antigen, and T cell-mediated specific killing of target-positive cells.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method including inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (cACT), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. No. 5,728,388, and International Publication No. WO 2008/081035.

The immune cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The term “engineered Autologous Cell Therapy,” which can be abbreviated as “cACT™,” also known as adoptive cell transfer, is a process by which a patient's own T cells are collected and subsequently genetically altered to recognize and target one or more antigens expressed on the cell surface of one or more specific tumor cells or malignancies. T cells can be engineered to express, for example, chimeric antigen receptors (CAR) or T cell receptor (TCR). CAR positive (+) T cells are engineered to express an extracellular single chain variable fragment (scFv) with specificity for a particular tumor antigen linked to an intracellular signaling part including at least one activating domain and in certain embodiments, at least one costimulatory domain. The costimulatory domain can be derived from (or correspond to), e.g., CD28, and the activating domain can be derived from (or correspond to) e.g., CD3-zeta. In certain embodiments, the CAR is designed to have two, three, four, or more costimulatory domains.

The term “autologous” refers to any material derived from the same individual to which it is later to be re-introduced. For example, the engineered autologous cell therapy (cACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

In some aspects, the disclosure therefore includes a method for treating or preventing a condition associated with undesired and/or elevated GPC3 levels in a patient, including administering to a patient in need thereof an effective amount of the cells as disclosed herein.

The cells of the disclosure can be used to treat various cancers. Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer.

In some embodiments, the cancer is one or more of hepatocellular carcinoma (HCC), lung squamous cell carcinoma, ovarian carcinoma, gastric carcinoma, melanoma, hepatoblastoma, nephroblastoma, Wilms tumor and a pediatric embryonal tumor. In a particular embodiment, the cancer is hepatocellular carcinoma (HCC).

It will be appreciated that target doses for the CAR-immune cells can range from 1×10⁶-2×10¹⁰ cells/kg, preferably 2×10⁶ cells/kg, more preferably. It will be appreciated that doses above and below this range may be appropriate for certain subjects, and appropriate dose levels can be determined by the healthcare provider as needed. Additionally, multiple doses of cells can be provided in accordance with the disclosure.

Also provided are methods for reducing the size of a tumor in a subject, including administering to the subject an engineered cell of the present disclosure to the subject, wherein the cell includes a chimeric antigen receptor, a T cell receptor, or a T cell receptor based chimeric antigen receptor including an antigen binding molecule binds to an antigen on the tumor. In some embodiments, the subject has a solid tumor, or a blood malignancy such as lymphoma or leukemia. In some embodiments, the engineered cell is delivered to a tumor bed. In some embodiments, the cancer is present in the bone marrow of the subject. In some embodiments, the engineered cells are autologous T cells. In some embodiments, the engineered cells are allogeneic T cells. In some embodiments, the engineered cells are heterologous T cells. In some embodiments, the engineered cells of the present application are transfected or transduced in vivo. In other embodiments, the engineered cells are transfected or transduced ex vivo. As used herein, the term “in vitro cell” refers to any cell which is cultured ex vivo. In particular, an in vitro cell can include a T cell.

The methods can further include administering one or more chemotherapeutic agent. In certain embodiments, the chemotherapeutic agent is a lymphodepleting (preconditioning) chemotherapeutic. In other embodiments, the engineered immune cells and the chemotherapeutic agent are administered each in an amount effective to treat the disease or condition in the subject.

In certain embodiments, compositions including CAR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, tricthylenethiophosphaoramide and trimethylolomelamine resume; nitrogen mustards such as chlorambucil, chlornaphazine, cholophosphamide, estramustine, ifosfamide, mechlorethaminc, mechlorethamine oxide hydrochloride, melphalan, novembichin, phenesterinc, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridinc, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; ctoglucid; gallium nitrate; hydroxyurca; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustinc; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifenc, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell, polypeptide, or nucleic acid. In other embodiments, the chemotherapeutic agent is administered from 1 to 4 weeks or from 1 week to 1 month, 1 week to 2 months, 1 week to 3 months, 1 week to 6 months, I week to 9 months, or 1 week to 12 months after the administration of the engineered cell, polypeptide, or nucleic acid. In other embodiments, the chemotherapeutic agent is administered at least 1 month before administering the cell, polypeptide, or nucleic acid. In some embodiments, the methods further include administering two or more chemotherapeutic agents.

A variety of additional therapeutic agents may be used in conjunction with the compositions described herein. For example, potentially useful additional therapeutic agents include PD-1 inhibitors such as nivolumab (Opdivo®), pembrolizumab (Keytruda®), pembrolizumab, pidilizumab, and atezolizumab.

Additional therapeutic agents suitable for use in combination with the disclosure include, but are not limited to, ibrutinib (Imbruvica®), ofatumumab (Arzerra®), rituximab (Rituxan®), bevacizumab (Avastin®), trastuzumab (Herceptin®), trastuzumab emtansine (KADCYLA®), imatinib (Gleevec®), cetuximab (Erbitux®), panitumumab (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimctinib, sclumetinib, trametinib, binimetinib, alectinib, ceritinib, crizotinib, aflibercept,adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### Methods of Manufacture

A variety of known techniques can be utilized in making the polynucleotides, polypeptides, vectors, antigen binding molecules, immune cells, compositions, and the like according to the disclosure.

Prior to the in vitro manipulation or genetic modification of the immune cells described herein, the cells may be obtained from a subject. In some embodiments, the immune cells include T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain embodiments, T cells can be obtained from a unit of blood collected from the subject using any number of techniques known to the skilled person, such as FICOLL™ separation. Cells may preferably be obtained from the circulating blood of an individual by apheresis. The apheresis product typically contains lymphocytes, including T cells, monocytes, granulocytes, B cells, other nucleated white blood cells, red blood cells, and platelets. In certain embodiments, the cells collected by apheresis may be washed to remove the plasma fraction, and placed in an appropriate buffer or media for subsequent processing. The cells may be washed with PBS. As will be appreciated, a washing step may be used, such as by using a semiautomated flowthrough centrifuge—for example, the Cobe™ 2991 cell processor, the Baxter CytoMate™, or the like. After washing, the cells may be resuspended in a variety of biocompatible buffers, or other saline solution with or without buffer. In certain embodiments, the undesired components of the apheresis sample may be removed.

In certain embodiments, T cells are isolated from PBMCs by lysing the red blood cells and depleting the monocytes, for example, using centrifugation through a PERCOLL™ gradient. A specific subpopulation of T cells, such as CD28+, CD4+, CD8+, CD45RA+, and CD45RO⁺ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present disclosure.

PBMCs may be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In some embodiments, CD8⁺ cells are further sorted into naive, central memory, and effector cells by identifying cell surface antigens that are associated with each of these types of CD8⁺ cells. In some embodiments, the expression of phenotypic markers of central memory T cells include CD45RO, CD62L, CCR7, CD28, CD3, and CD127 and are negative for granzyme B. In some embodiments, central memory T cells are CD45RO⁺, CD62L⁺, CD8⁺ T cells. In some embodiments, effector T cells are negative for CD62L, CCR7, CD28, and CD127, and positive for granzyme B and perforin. In certain embodiments, CD4⁺ T cells are further sorted into subpopulations. For example, CD4⁺ T helper cells can be sorted into naive, central memory, and effector cells by identifying cell populations that have cell surface antigens.

The immune cells, such as T cells, can be genetically modified following isolation using known methods, or the immune cells can be activated and expanded (or differentiated in the case of progenitors) in vitro prior to being genetically modified. In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, for example, in U.S. Pat. Nos. 6,905,874; 6,867,041; 6,797,514; and PCT WO2012/079000, the contents of which are hereby incorporated by reference in their entirety. Generally, such methods include contacting PBMC or isolated T cells with a stimulatory molecule and a costimulatory molecule, such as anti-CD3 and anti-CD28 antibodies, generally attached to a bead or other surface, in a culture medium with appropriate cytokines, such as IL-2. In certain embodiments, such methods include culturing cells in medium comprising exogenous IL-7 and exogenous IL-21, in the absence of exogenous IL-2. In certain aspects, CD4 and CD8⁺ cells are isolated and/or enriched from peripheral blood mononuclear cells, activated with anti-CD3 and anti-CD28 antibodies, and grown in medium supplemented with exogenous IL-7 and exogenous IL-21, but not exogenous IL-2. In certain further aspects, cells can be expanded, post-transduction, for a selected amount of time prior to harvest including but not limited to 1 day, 2 days, 3 days, 4 days, 5 days, 6 days, 7 days, 8 days, 9 days or 10 days. Preferably, cells are harvested 3 days, 5 days or 8 days post-transduction. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other embodiments, the T cells may be activated and stimulated to proliferate with feeder cells and appropriate antibodies and cytokines using methods such as those described in U.S. Pat. Nos. 6,040,177; 5,827,642; and WO2012129514, the contents of which are hereby incorporated by reference in their entirety.

Certain methods for making the constructs and engineered immune cells of the disclosure are described in PCT application PCT/US15/14520, the contents of which are hereby incorporated by reference in their entirety. Additional methods of making the constructs and cells can be found in U.S. Pat. No. 11,723,923 the contents of which are hereby incorporated by reference in its entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum.

For cloning of polynucleotides, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, cubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the disclosure provides a method of storing genetically engineered cells expressing CARs or TCRs which target a GPC3 protein. This involves cryopreserving the immune cells such that the cells remain viable upon thawing. A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

Desired treatment amounts of cells in the composition are generally at least 2 cells (for example, at least 1 CD8⁺ central memory T cell and at least 1 CD4⁺ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹ , or 10¹² cells. In some aspects of the present disclosure, particularly since all the infused cells will be redirected to a particular target antigen (GPC3), lower numbers of cells, in the range of 10⁶/kilogram (10⁶-10¹¹ per patient) may be administered. CAR treatments may be administered multiple times at dosages within these ranges. The cells may be autologous, allogeneic, or heterologous to the patient undergoing therapy.

The CAR expressing cell populations of the present disclosure may be administered either alone, or as a pharmaceutical composition in combination with diluents and/or with other components such as IL-2 or other cytokines or cell populations. Pharmaceutical compositions of the present disclosure may include a CAR or TCR expressing cell population, such as T cells, as described herein, in combination with one or more pharmaceutically or physiologically acceptable carriers, diluents or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), may include one or more of the following: sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

It will be appreciated that adverse events may be minimized by transducing the immune cells (containing one or more CARs or TCRs) with a suicide gene. It may also be desired to incorporate an inducible “on” or “accelerator” switch into the immune cells. Suitable techniques include use of inducible caspase-9 (U.S. Appl. 2011/0286980) or a thymidine kinase, before, after or at the same time, as the cells are transduced with the CAR construct of the present disclosure. Additional methods for introducing suicide genes and/or “on” switches include TALENS, zinc fingers, RNAi, siRNA, shRNA, antisense technology, and other techniques known in the art.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. Sec, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

The following examples, including the experiments conducted and results achieved, are provided for illustrative purposes only and are not to be construed as limiting the present disclosure.

### EXAMPLES

### Example 1. Preparation and Expression of Tricistronic Vectors

Engineered GPC3 CAR T cells expressing an engineered, dominant-negative TGF-β receptor (TGFβRII DNR, or simply TGFβ DNR) and an engineered, membrane bound IL-15 tethered to IL-15Ra agonist receptor (mbIL15) were tested in three different architectures with an EFla promoter (SEQ ID NO: 1) as described below and illustrated in FIG. 1.

The first architecture, referred to herein as “Tri-2A,” included a coding sequence for GPC3 CAR (SEQ ID NO:14 upon removal of the signal peptide), a coding sequence for TGFβ DNR (SEQ ID NO:8), and a coding sequence for mbIL15 (SEQ ID NO:10). A coding sequence for T2A self-cleaving peptide (SEQ ID NO:2) was placed between the GPC3 CAR and TGFβ DNR coding sequences. Further, a coding sequence for P2A self-cleaving peptide (SEQ ID NO: 3) was placed between the TGFβ DNR and mbIL 15 coding sequences.

The second architecture, referred to herein as “Tri-IRES,” differs from Tri-2A by substituting the coding sequence for P2A self-cleaving peptide in Tri-2A with a coding sequence for Encephalomyocarditis virus (EMCV) internal ribosome entry site (IRES) (SEQ ID NO: 5).

The third architecture, referred to herein as “Tri-mSCV,” differs from Tri-2A by substituting the coding sequence for P2A self-cleaving peptide in Tri-2A with a coding sequence for murine Stem Cell Virus (mSCV) IRES (SEQ ID NO: 6), followed by a SP163 translational enhancer (SEQ ID NO:7) derived from the 5′UTR of the murine VEGF gene.

Also tested were two control constructs, “CAR-DNR” (GPC3 CAR fused to TGFβ DNR through a T2A peptide), and “CAR-mbIL15” (GPC3 CAR fused to mbIL 15 through a T2A peptide).

A lentivirus vector was used for all T-cell transductions. CD4 and CD8⁺ cells were isolated from healthy donor peripheral blood mononuclear cells using a CliniMACS Prodigy and frozen down in CryoStor® cell cryopreservation media (Sigma Aldrich®). Before lentivirus transduction, T cells were thawed, activated with plate bound CD3 (Miltenyi Biotec) and soluble CD28 (BD Biosciences) and rested overnight. The following day cells were transduced with lentivirus containing tricistronic CAR constructs as described herein. Cells were grown for 8 days in OpTmizer Media with T-cell expansion supplement, Immune Cell SR and Glutamax (Gibco), supplemented two times per week with 10 μg/L IL7 and 10 μg/L IL21 (Peprotech). After the 8-day cell culture, cells in all trials were approximately 30-80% positive for GPC3 CAR expression as measured by flow cytometry with an average vector copy number of approximately 0.5 to 3. Cells used in the functional assays and in vivo experiments described below were performed after thawing and overnight rest with 10 μg/L of IL7 and 10 μg/L IL21.

T cells transduced with CAR-DNR, CAR-mbIL15, Tri-2A, Tri-IRES, Tri-mSCV, and nontransduced (NTD) were produced from 3-5 healthy donors. Vector copy number (VCN) of these cells was measured via ddPCR. The measured VCN are shown in FIG. 2 and Table 1. As shown, all tested vectors exhibited high copy numbers. Importantly, while the tricistronic vectors were considerably larger than bicistronic counterparts, their VCN were only slightly lower.

Surface expression of CAR, TGFβ DNR, and mbIL15 on these cells was measured by flow cytometry. MFI was normalized to CAR and TGFβ DNR of CAR-DNR cells and mbIL15 of CAR-mbIL 15 cells. As shown in FIG. 3 and Table 1, all of the proteins in all T cells transduced with both the tricistronic vectors and the bicistronic vectors exhibited high expression. Significantly, in T cells transduced with Tri-IRES, the expression levels of GPC3 CAR, TGFβ DNR, and mbIL15 were substantially equimolar.

TABLE 1
Expression and copy numbers of the constructs

| | | | | Copy |
|---|---|---|---|---|
| Construct | % CAR+ | % DNR+ | % mbIL15+ | Number |
| Tri-2A | 48.7 | 16.4 | 50.2 | 0.9 |
| Tri-IRES | 32.4 | 33.5 | 27.4 | 0.7 |
| Tri-mSCV | 36.7 | 38.8 | 10.5 | 0.7 |
| CAR-DNR | 64.3 | 67.1 | 0.0 | 1.6 |
| CAR- | 54.4 | 0.1 | 59.2 | 1.2 |
| mbIL15 | | | | |
| NTD | 0.1 | 0.1 | 0.1 | 0.0 |

### Example 2. Cytotoxicity and Cytokine Secretion

The cytotoxicity of the CAR T cells manufactured as described in Example 1 was evaluated in a xCELLigence RTCA assay on an MP instrument (Agilent) in three (Tri-mSCV) to five (Tri-2A and Tri-IRES) independent donors. 10,000 live target cells (GPC3 negative: SKHep1; GPC3 low/medium: Huh7 and GPC3 high: Hep3B) were plated the day prior to the assay, and on the next day, CAR+ T cells (or non-transduced (NTD)) were added for an Effector-to-Target (E: T) ratio of 1:1, 1:5 and 1:20 (10,000, 2,000, and 500 CAR+ T cells, respectively) were plated in a 96-well E-plate in target cell culture media, following the manufacturer's recommendations. Cytotoxicity was followed over 120 hours using the RTCA Pro software provided and is reported as % cytolysis. Data is shown for one representative donor at an E: T of 1:5 (SKHep1, Huh7) or 1:20 (Hep3B) manufactured as described in Example 1.

Cytokine secretion by TGFβ DNR and mbIL15-enhanced GPC3-specific CAR T cells was evaluated after a coculture assay. 25,000 target cells and 25,000 CAR+ T or non-transduced (NTD) cells were plated in a flat bottom 96-well plate and in target cell culture media. After 24h, supernatants were collected, diluted 50 or 500 times, and IL-2 and IFNγ were measured by Milliplex MAP kit (EMD Millipore Sigma) according to manufacturer's directions. Target cell lines used were a GPC3 negative line, SKHep; a GPC3 low/medium line, Huh7; and a GPC3 high line, Hep3B. This experiment was run with cells from 3-5 healthy donors, and the data in FIG. 4 and Table 2 is showing one representative donor.

TABLE 2
Cytotoxicity and cytokine production of the constructs

| Effector:Target | | | | | | | | | |
|---|---|---|---|---|---|---|---|---|---|
| (E:T) ratio | High E:T | | | Med E:T | | | Low E:T | | |
| Target cell | 10,000 | 10,000 | 10,000 | 10,000 | 10,000 | 10,000 | 10,000 | 10,000 | 10,000 |
| number | | | | | | | | | |
| seeded | | | | | | | | | |
| Target cell | SKHep1 | Huh7 | Hep3B | SKHep1 | Huh7 | Hep3B | SKHep1 | Huh7 | Hep3B |
| CAR T cell | 10,000 | 2000 | 10,000 | 2000 | 2000 | 2000 | 500 | 500 | 500 |
| number added | | | | | | | | | |
| | % Cytolysis (96 h post CAR T addition) | | | | | | | | |
| Tri-2A | 25.8 | 98.3 | 100.0 | 9.6 | 40.6 | 93.7 | 9.2 | 5.1 | 55.0 |
| Tri-IRES | 34.7 | 100.0 | 100.0 | 3.3 | 62.6 | 97.3 | 0.0 | 17.3 | 71.5 |
| Tri-mSCV | 28.2 | 100.0 | 100.0 | 11.5 | 68.7 | 93.0 | 8.1 | 16.7 | 77.4 |
| CAR-DNR | 17.7 | 94.4 | 100.0 | 2.8 | 48.5 | 96.3 | 0.0 | 4.8 | 68.7 |
| CAR-mbIL15 | 35.9 | 98.3 | 100.0 | 11.4 | 45.7 | 91.2 | 5.4 | 18.4 | 41.5 |
| NTD | 28.5 | 17.9 | 29.8 | 13.7 | 14.9 | 3.1 | 18.6 | 20.9 | 2.3 |

| Cytokine | IL-2 | | | IFNγ | | |
|---|---|---|---|---|---|---|
| Target cell number | 10,000 | 10,000 | 10,000 | 10,000 | 10,000 | 10,000 |
| seeded | | | | | | |
| Target cell | SKHep1 | Huh7 | Hep3B | SKHep1 | Huh7 | Hep3B |
| CAR T cell number | 10,000 | 10,000 | 10,000 | 10,000 | 10,000 | 10,000 |
| added | | | | | | |

Cytokine (24 h) pg/ml

| | | | | | | |
|---|---|---|---|---|---|---|
| Tri-2A | 5.0 | 48.7 | 1486.1 | 372.8 | 17217.4 | 62729.2 |
| Tri-IRES | 5.2 | 300.0 | 3766.8 | 245.6 | 33335.0 | 57111.9 |
| Tri-mSCV | 5.1 | 42.1 | 1375.4 | 355.9 | 33611.6 | 71150.6 |
| CAR-DNR | 4.8 | 17.6 | 752.9 | 376.5 | 26933.7 | 72075.8 |
| CAR-mbIL15 | 5.5 | 31.1 | 367.4 | 875.9 | 10659.7 | 38639.2 |
| NTD | 5.0 | 5.0 | 4.9 | 288.0 | 66.6 | 71.4 |

As shown in the figure and table, cytotoxicity of the tricistronic constructs matched or exceeded the cytotoxicity of the bicistronic ones. In particular, all of the tricistronic constructs stimulated higher IL-2 secretion than the bicistronic ones, and all of them had similar IFNγ secretion.

### Example 3. Cytokine Independent Growth Assays

To investigate whether expressing the membrane-bound IL-15 chimeric receptors could enhance persistence of tricistronic CARs, the constructs described above were evaluated in a cytokine independent growth assay. On day 0, 500,000 CAR T cells plus NTD cells to normalize total cell numbers were seeded in RPMI+10% FBS media (Gibco) with no exogenous cytokine. Media was exchanged every 3-5 days and cells were counted at each timepoint by flow cytometry.

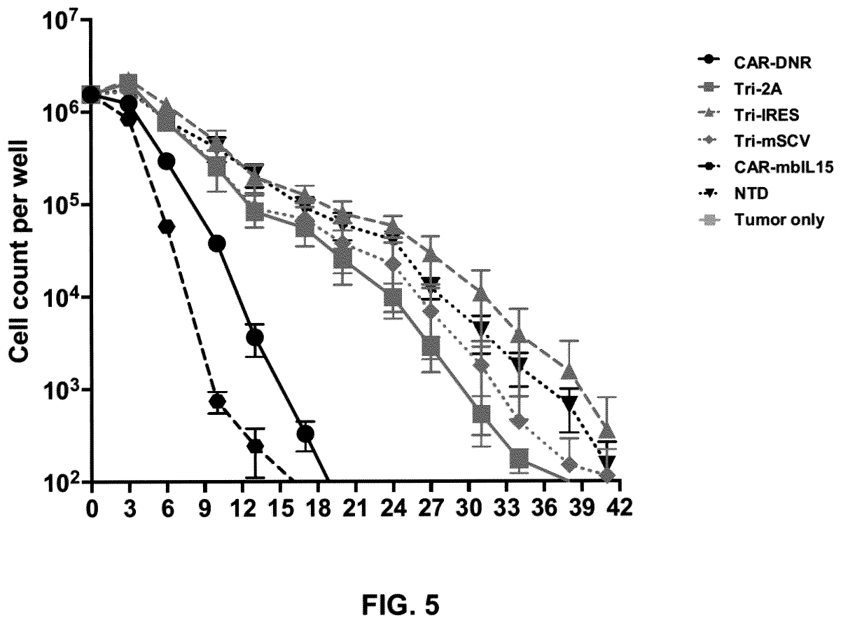

The data shown in FIG. 5 and Table 3 is from a representative donor. Constructs expressing mbIL15 (Tri-IRES, Tri-2A, Tri-mSCV, and CAR-mbIL15) persisted significantly longer than NTD and CAR-DNR cells, which do not express mbIL15, demonstrating the survival benefit of mbIL 15 enhancement in the tricistronic CARs.

TABLE 3
Cytokine independent growth assay results

| | Day 0 |
|---|---|
| CAR T cells per | 500000 |
| well | |
| % CAR Normalized | 32.4% |

| | CD3+ Cell Count (cells/μl) | | | | | |
|---|---|---|---|---|---|---|
| Day | 3 | 6 | 10 | 13 | 17 | 20 |
| Tri-2A | 2052986 | 773280 | 250949 | 82931 | 55074 | 25225 |
| Tri-IRES | 2233747 | 1183224 | 472918 | 197564 | 125297 | 78992 |
| Tri-mSCV | 1662523 | 843962 | 258237 | 90422 | 69276 | 37668 |
| CAR-DNR | 1231512 | 293974 | 37905 | 3645 | 330 | 48 |
| CAR-mbIL15 | 1734504 | 803628 | 407776 | 209675 | 91735 | 60164 |
| NTD | 836265 | 57584 | 743 | 244 | 77 | 19 |

| Day | 24 | 27 | 31 | 34 | 38 | 41 |
|---|---|---|---|---|---|---|
| Tri-2A | 9795 | 2856 | 534 | 172 | 99 | 26 |
| Tri-IRES | 58336 | 29030 | 10946 | 3887 | 1554 | 360 |
| Tri-mSCV | 22381 | 6875 | 1792 | 444 | 151 | 115 |
| CAR-DNR | 48 | 9 | 10 | 0 | 0 | 0 |
| CAR-mbIL15 | 41042 | 12469 | 4277 | 1738 | 666 | 150 |
| NTD | 45 | 10 | 0 | 0 | 0 | 9 |

### Example 4. Suppression of SMAD2 and SMAD3 Phosphorylation

500,000 CAR T cells plus NTD cells to normalize total cell numbers were cultured in RPMI media without serum for two hours before 30-minute stimulation with 5 ng/mL of soluble recombinant TGF-β1 that was reconstituted in acid as recommended by the manufacturer (R&D Systems). Cells were then collected by centrifugation and lysed with RIPA buffer+protease and phosphatase inhibitors (ThermoFisher Scientific) before LUMINEX® analysis for pSMAD2 and pSMAD3. All tricistronic constructs except Tri-mSCV were run in three donors.

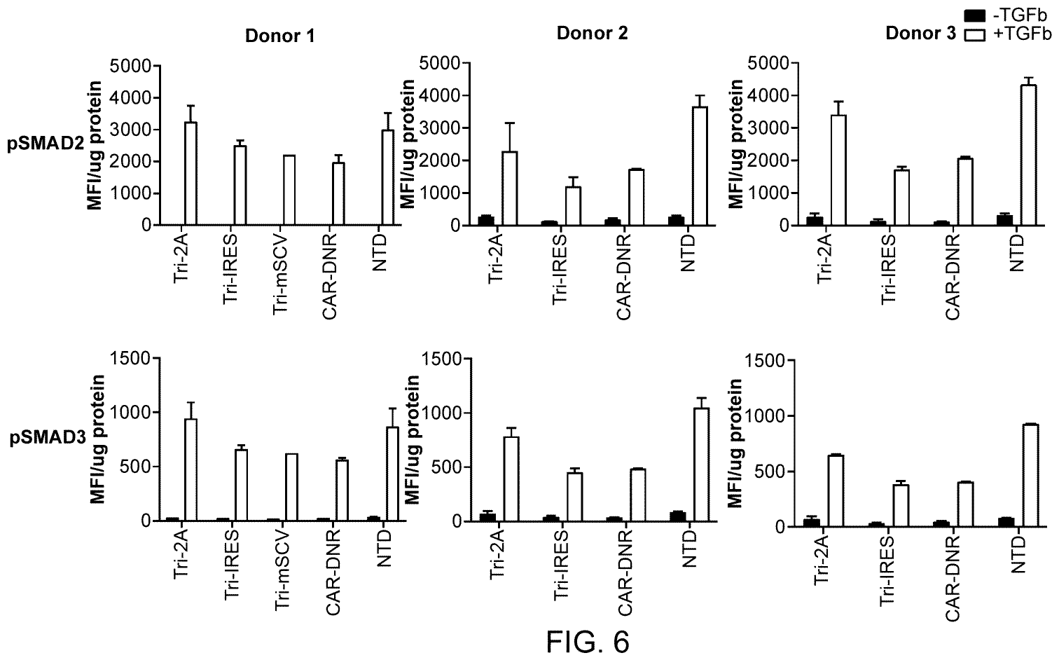

The data (FIG. 6, Table 4) suggests that the truncated form TGFβ DNR is functional in tricistronic CARs, blocking signal transduction and limiting the inhibitory pSMAD signaling induced by TGF-β.

TABLE 4
SMAD phosphorylation

| | Donor 1 | | Donor 2 | | Donor 3 | |
|---|---|---|---|---|---|---|
| TGFβ | − | + | − | + | − | + |
| CAR T cells | 500000 | 500000 | 500000 | 500000 | 500000 | 500000 |
| % CAR | 32.4 | 32.4 | 47.2 | 47.2 | 47.2 | 47.2 |
| Normalized | | | | | | |
| | pSMAD2 (MFI/μg protein) | | | | | |
| Tri-2A | 19.0 | 1969.8 | 118.9 | 2077.2 | 168.2 | 1734.7 |
| Tri-IRES | 12.6 | 3240.5 | 246.5 | 3396.3 | 235.3 | 2268.0 |
| Tri-mSCV | 18.2 | 2484.1 | 131.5 | 1718.8 | 108.5 | 1202.5 |
| CAR-DNR | 7.9 | 2214.6 | — | — | — | — |
| NTD | 29.3 | 2976.3 | 315.2 | 4324.7 | 269.7 | 3625.5 |
| | pSMAD3 (MFI/μg protein) | | | | | |
| Tri-2A | 19.4 | 561.1 | 31.8 | 487.7 | 44.0 | 405.1 |
| Tri-IRES | 21.1 | 942.0 | 65.1 | 787.9 | 68.1 | 642.9 |
| Tri-mSCV | 15.5 | 659.6 | 40.1 | 459.5 | 29.1 | 377.4 |
| CAR-DNR | 11.5 | 620.8 | — | — | — | — |
| NTD | 33.5 | 866.3 | 88.2 | 1047.4 | 74.1 | 917.6 |

### Example 5. In vivo and ex vivo Testing

The in vivo study was performed to assess and compare the antitumor activity of different CAR constructs expressing a TGFβ dominant negative receptor (DNR) and mbIL15 in comparison to CAR+DNR or CAR+mbIL15 in a xenograft Hep3B subcutaneous mouse model.

T-cell products were manufactured and cryopreserved, and consisted of non-transduced (NTD) T cells, T cells transduced with CAR and DNR only (CAR-DNR), CAR and mbIL-15 only (CAR-mbIL15), CAR, DNR and mbIL15 in either the Tri-2A, Tri-IRES, or Tri-mSCV architectures. T cells were then tested in a subcutaneous Hep3B mouse model.

Briefly, Hep3B tumor cells (ATCC) were admixed 50%/50% in PBS and Matrigel (Corning, #354234) and implanted subcutaneously in the right dorsal flank of 10-week-old NSG-MHC I/II DKO female mice (The Jackson Laboratory, #025216). Mice were randomized on tumor volume (calculated as: width2*length)/2) when tumors reach ˜150 mm³ and assigned to groups of 8 mice. NTD and CAR T cells were thawed on the same day and rested overnight. Treatment was administered on the following day which consisted of an intravenous injection of NTD or CAR T cells. Body weight and tumors were measured twice a week, using a scale and a caliper, respectively. Body weight for each cohort is expressed as change from initial body weight. Blood samples were collected 24h post-T-cell infusion and weekly thereafter to analyze CAR+ cells/μl blood by flow cytometry using fluorochrome-conjugated antibodies and CountBright™ Absolute Counting beads (Invitrogen, #C36950). Mean tumor volume, mean body weight and mean CAR+ cells/μl blood values are only represented when ≥67% of mice remained in each cohort. Supportive care (ClearH20, DietGel® 93M) was provided when mice lost >10% body weight. End-of-life endpoints were defined by tumor volume reaching ˜ 2000 mm³, clinical observations, and body weight loss >20%.

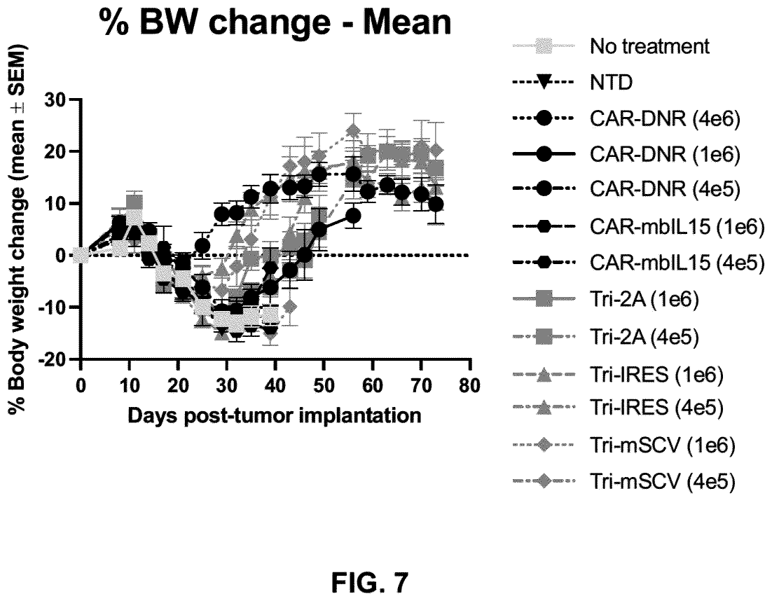

All mice experienced body weight loss early on followed by weight gain for the mice that achieved tumor control (FIG. 7).

Based on tumor burden, treatment with the tricistronic constructs were more potent than the bicistronic ones at the 1e6 (1×10⁶) CAR dose, with 4 to 7 out of 8 mice with a tumor volume <226 mm³ in the tricistronic constructs groups vs 1 to 4 out of 8 mice left by study end in the bicistronic groups, and with a tumor volume >495 mm³, on the last day of the study (Day 73) (FIG. 8A-B). At this dose, all animals but two treated with CAR-mbIL15 died within 50 days; many that were treated with CAR-DNR also did not survive through the end of the study; while the vast majority treated with the tricistronic constructs lived through the entire study and became almost tumor free.

At the lower 4e5 CAR dose, mice that were treated with the Tri-2A or Tri-IRES tricistronic constructs experienced tumor control, whereas all the other groups, no treatment, NTD, CAR-DNR and CAR-mbIL-15, demonstrated no efficacy and none of the animals in these groups lived till the end of the study (FIG. 8A-B).

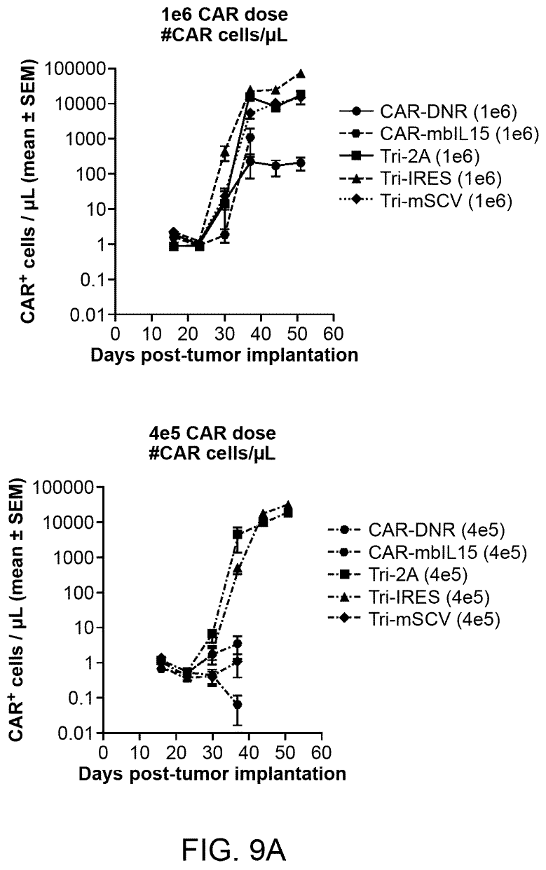

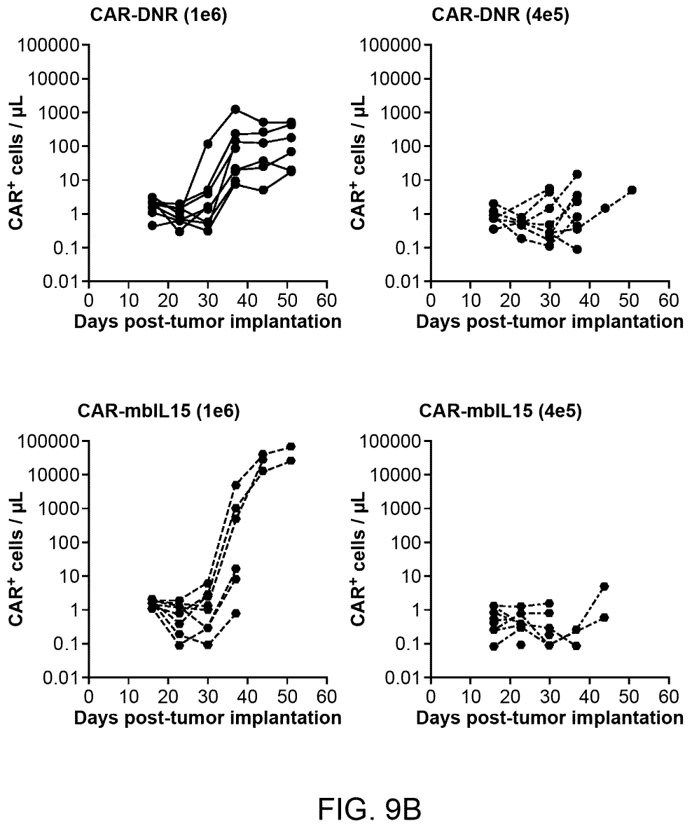

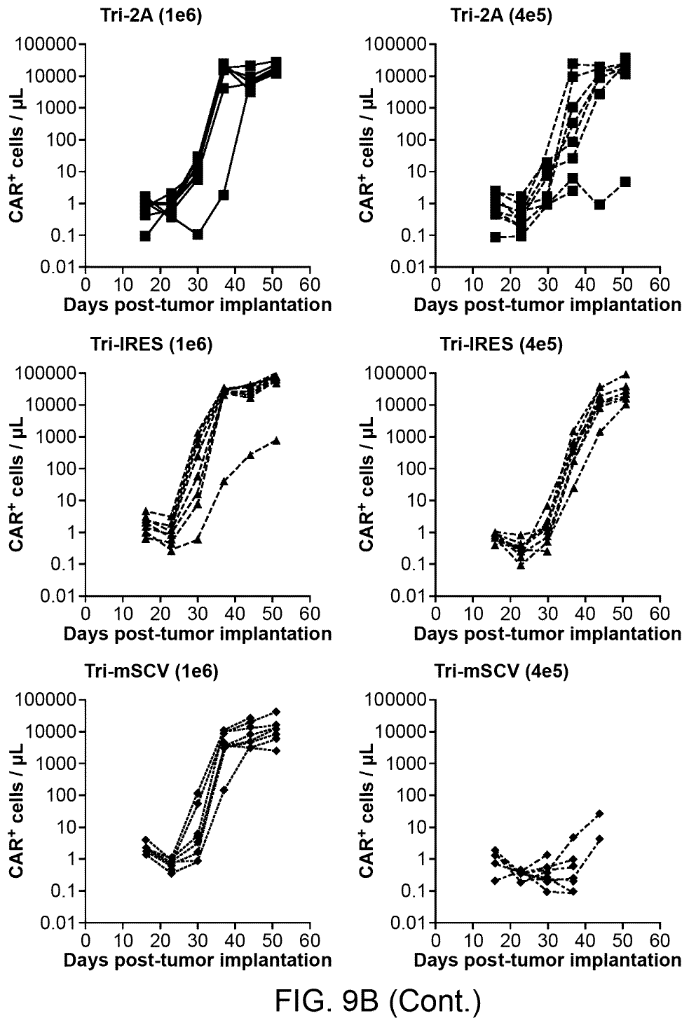

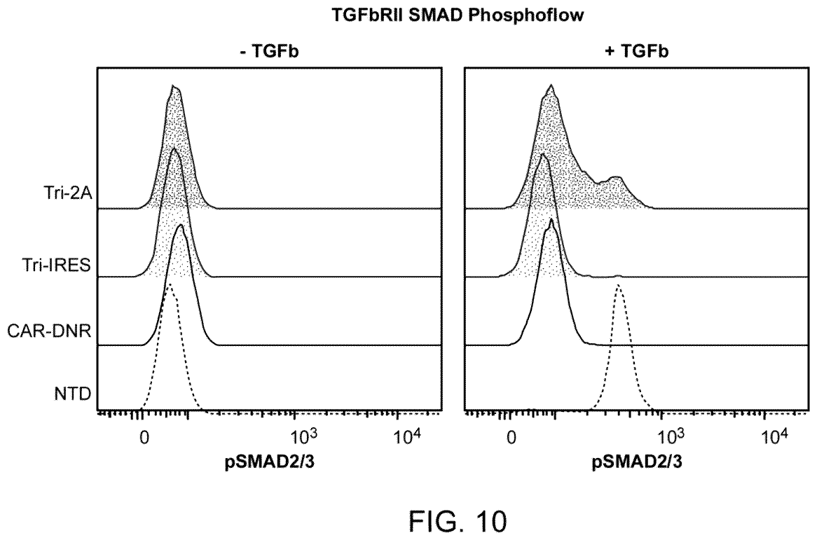

In the peripheral blood, tricistronic constructs achieved greater CAR+ T-cell expansion and persistence than the bicistronic ones, at both the 1e6 and 4e5 CAR doses (FIG. 9A-B).

### Example 6. TGFbeta DNR Phosphoflow

500,000 T cells were cultured in RPMI media without serum for two hours before 30-minute stimulation with 5 ng/ml of soluble recombinant TGF-β1 that was reconstituted in acid as recommended by the manufacturer (R&D Systems). Cells were then fixed and stained for CAR and DNR. Following surface staining, cells were permeabilized, stained for pSMAD2/3, and expression of pSMAD 2/3 on CAR+ cells was measured by flow cytometry. All constructs were run in three donors. The data (FIG. 10, Table 5) further confirm that the expression of the truncated TGF-β DNR in tricistronic CARs is sufficient to block signal transduction and limit the inhibitory pSMAD signaling induced by TGF-β.

TABLE 10
TGFbRII SMAD Phosphoflow

| | Donor 1 | | Donor 2 | | Donor 3 | |
|---|---|---|---|---|---|---|
| TGFb | − | + | − | + | − | + |
| | pSMAD2/3 (MFI CAR+) | | | | | |
| Tri-2A | 71.0 | 105.7 | 102.0 | 208.3 | 104.3 | 189.3 |
| Tri-IRES | 70.3 | 70.6 | 89.3 | 89.7 | 87.5 | 89.4 |
| CAR-DNR | 81.9 | 86.9 | 107.0 | 105.3 | 107.0 | 106.3 |
| NTD* | 59.7 | 372.0 | 75.3 | 495.7 | 79.1 | 413.7 |

*pSMAD2/3 MFI of CD3+ shown for NTD

### Example 7. In vivo and ex vivo Testing with Tumor Rechallenge

The in vivo study was performed to assess and compare the antitumor activity of different CAR constructs expressing a TGFβ dominant negative receptor (DNR) and mbIL15 in comparison to CAR+DNR in a xenograft Hep3B subcutaneous mouse model with tumor rechallenge.

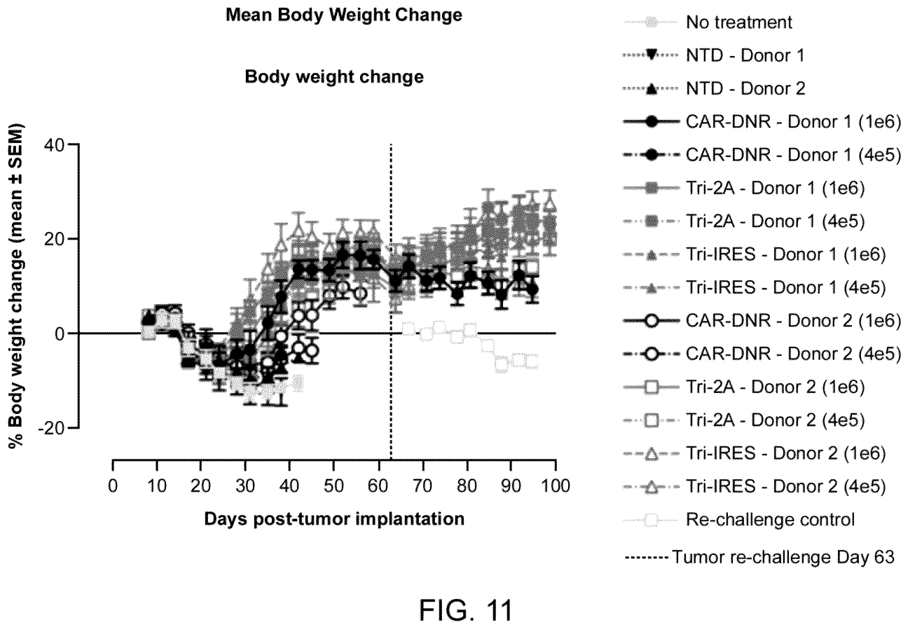

T-cell products from two donors were manufactured and cryopreserved and consisted of non-transduced (NTD) T cells, T cells transduced with CAR and DNR only (CAR-DNR), CAR, DNR and mbIL15 in either the Tri-2A or Tri-IRES. T cells were then tested in a subcutaneous Hep3B mouse model. The in vivo study protocol was as described in Example 5 with the following addition: All mice experienced body weight loss early on followed by weight gain in response to supportive care and tumor control (FIG. 11).

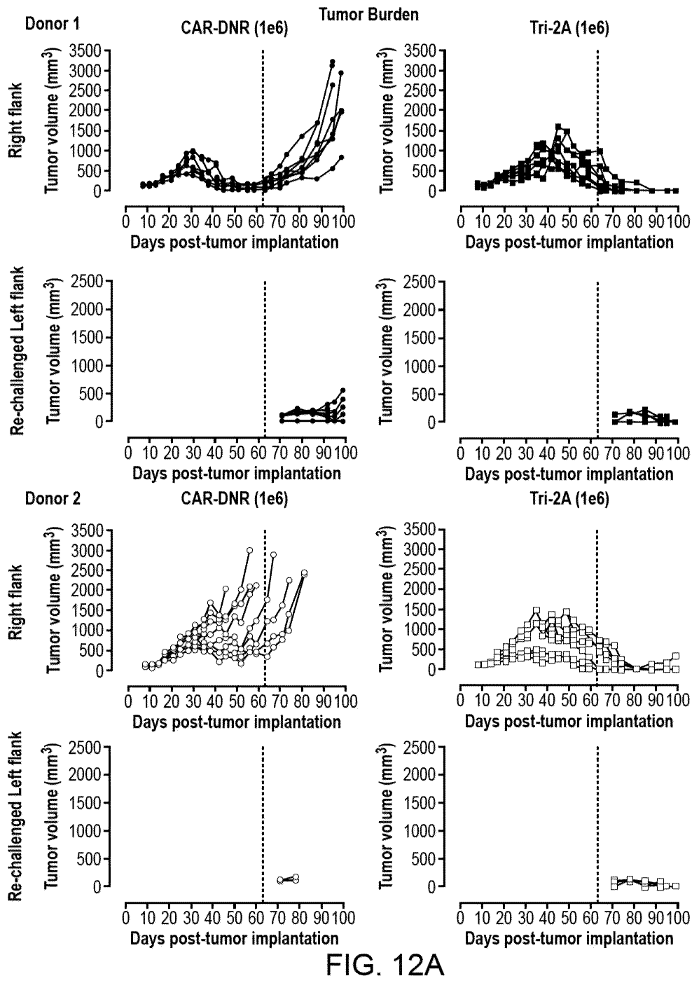

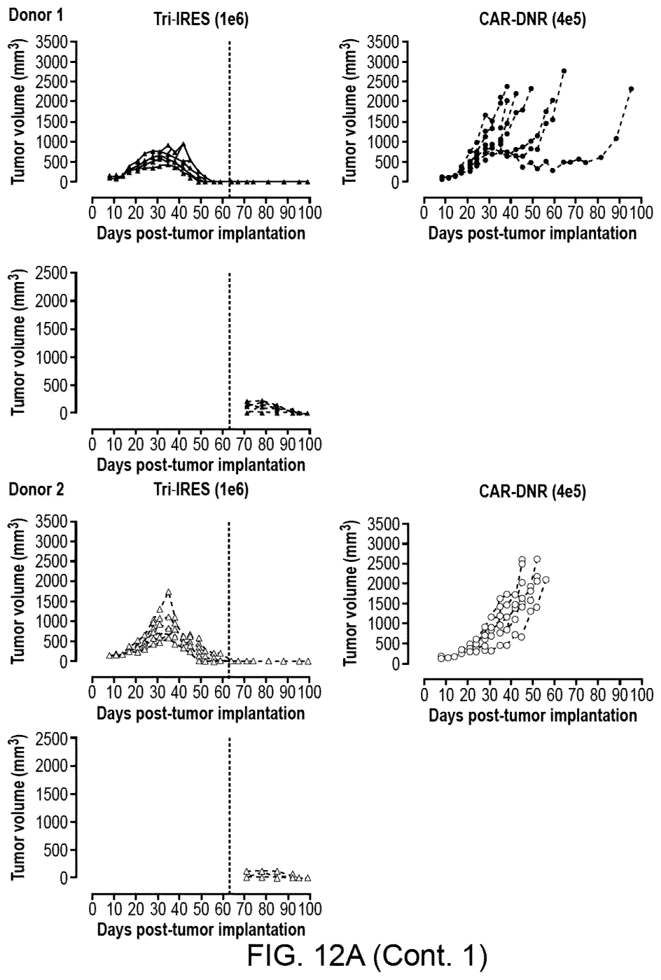

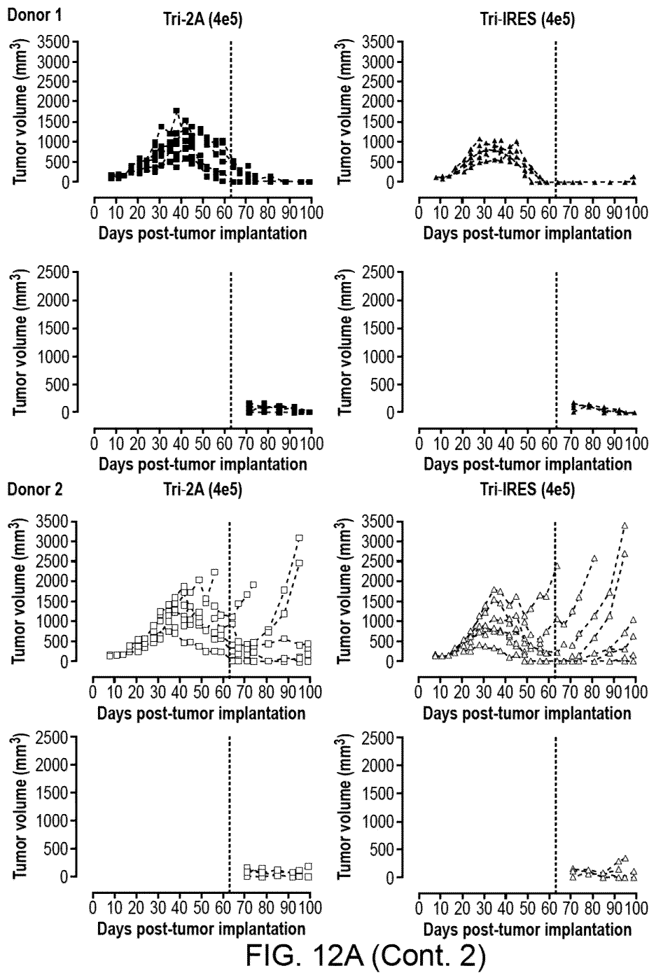

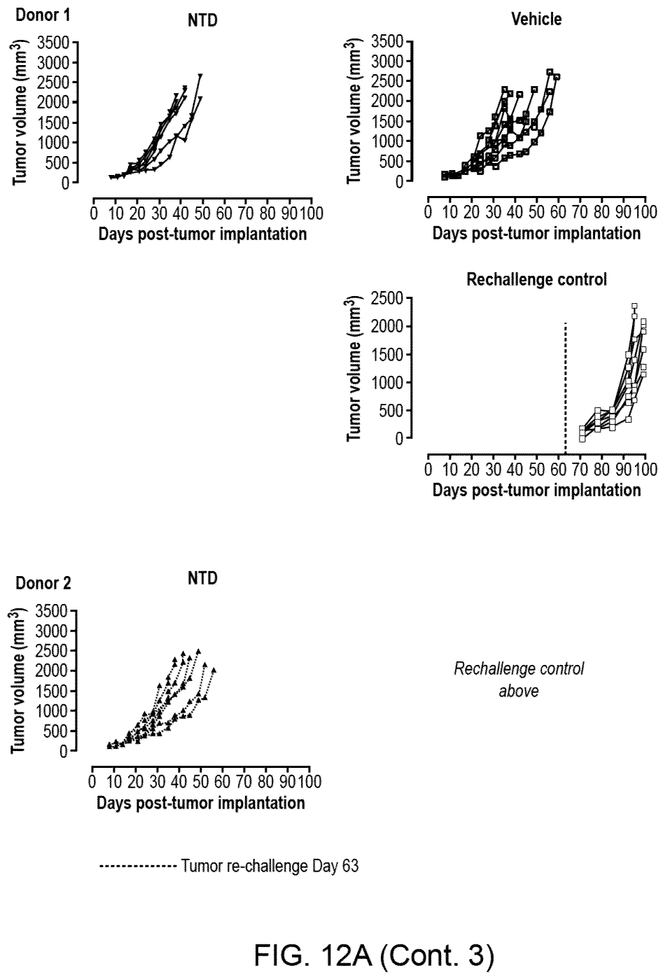

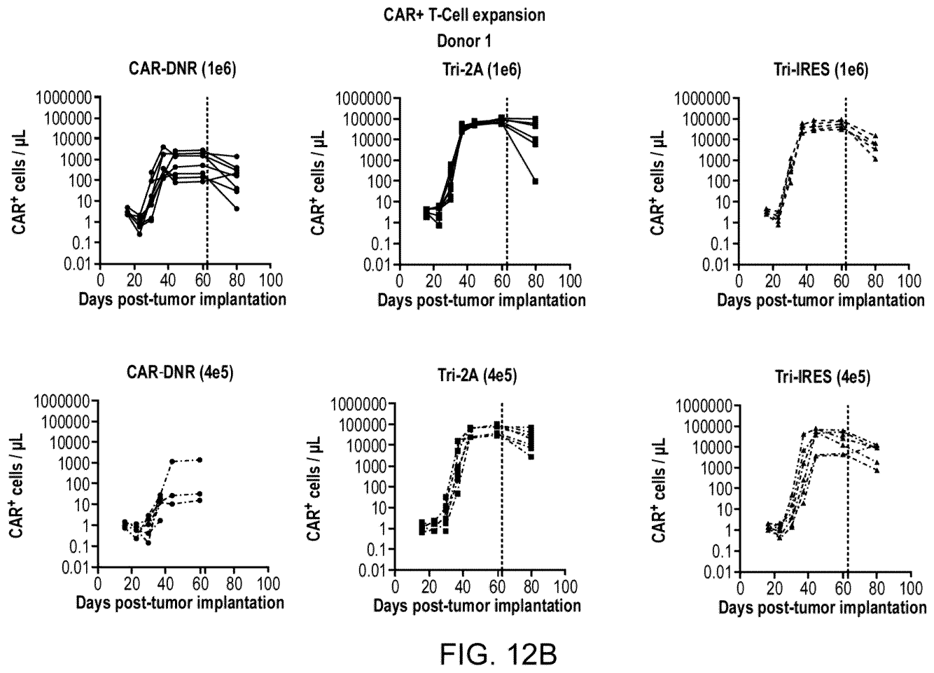

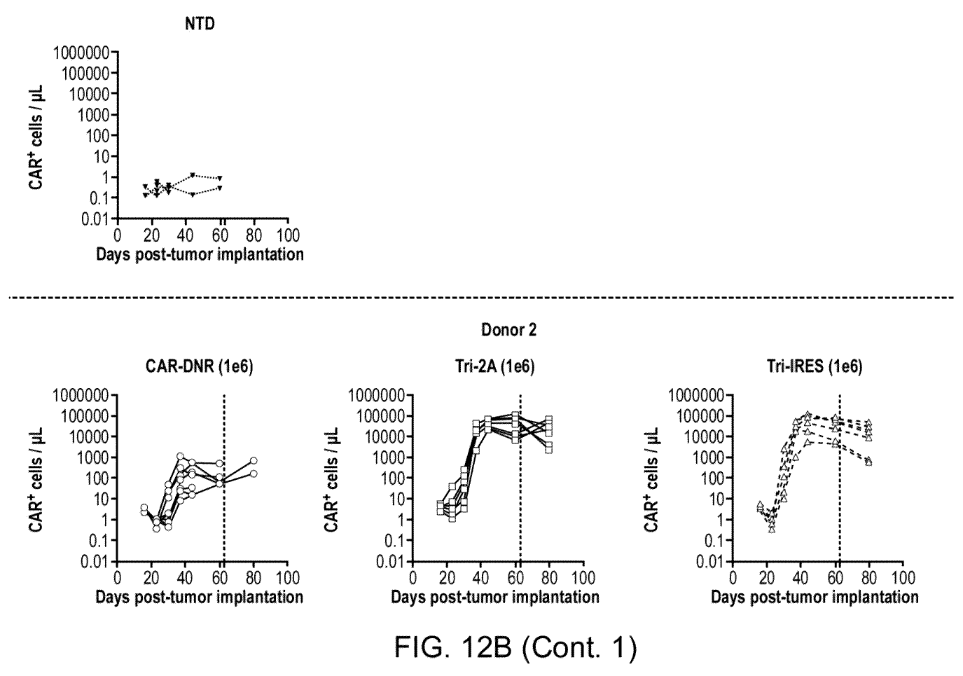

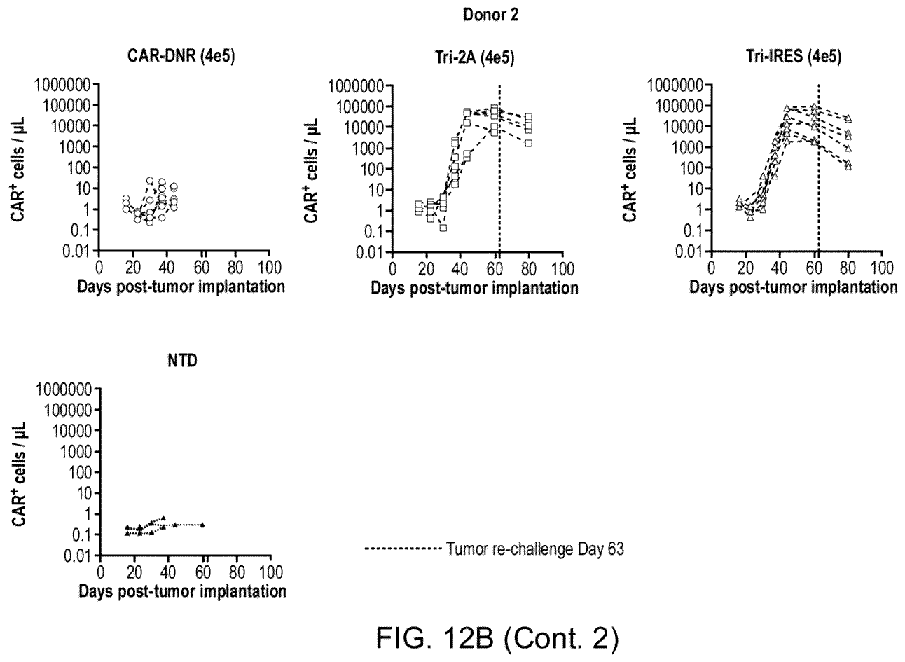

Based on tumor burden, treatment with the tricistronic constructs was more potent than the bicistronic one at the 1e6 (1×10⁶) CAR dose with complete tumor control achieved prior to and after tumor rechallenge with the tricistronic constructs and tumor control followed by relapse with the bicistronic construct, by study end, in Donors 1 and 2. Following tumor rechallenge in the opposite flank, mice treated with tricistronic CAR maintained control of both the primary tumor and the secondary tumor (rechallenge), while mice treated with bicistronic CAR had rapid tumor relapse in the primary tumor and signs of tumor outgrowth in the secondary tumor. (FIG. 12A-12B).

At the lower 4e5 CAR dose, mice treated with the tricistronic constructs from Donor 1 showed complete tumor control prior to and after tumor rechallenge, while the other groups, no treatment, NTD and CAR-DNR, demonstrated no efficacy, with only 1 of 8 mice in the CAR-DNR group in one donor that showed tumor control followed by relapse by the end of the study. For Donor 2, with Tri-IRES, tumor control was observed in 6 out of 8 mice prior to rechallenge, followed by tumor relapse in 5 out of 6 rechallenged mice, and complete tumor control in 1 out of 6 rechallenged mice, while for Tri-2A, 2 out of 8 mice showed some tumor control followed by relapse and were excluded from tumor rechallenge, with from the 6 rechallenged, 3 out of 6 showed signs of relapse by study end and 3 out of 6 showed controlled tumor by study end. (FIG. 12A-B).

In the peripheral blood, the tricistronic constructs had greater CAR+ T-cell expansion and persistence compared to the bicistronic CAR at both the 1e6 and 4e5 CAR doses (FIG. 12A-B).

### Example 8. Early Harvest

The in vivo study was performed to assess and compare the antitumor activity of Day 3, Day 5, and Day 8 harvested CAR T cells expressing a TGFb dominant negative receptor (DNR) and mbIL15 in comparison to Day 8 harvested CAR+DNR in a xenograft Hep3B subcutaneous mouse model.

T-cell products were manufactured and cryopreserved on day 3, day 5 or day 8 and consisted of non-transduced (NTD) T cells, T cells transduced with CAR and DNR only (CAR-DNR), CAR and mbIL-15 only (CAR-mbIL15), CAR, DNR and mbIL 15 in either the Tri-2A or Tri-IRES. T cells were then tested in a subcutaneous Hep3B mouse model.

Hep3B tumor cells were implanted subcutaneously in the right dorsal flank of 8/9-week-old NSG-MHC I/II DKO female mice (The Jackson Laboratory, #025216) and in vivo study protocol and measurements were as described in Example 5.

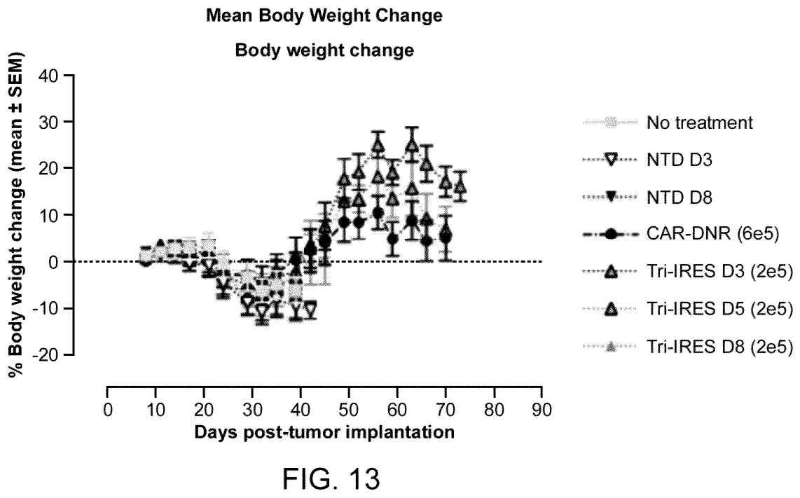

Some mice experienced weight loss early in the study followed by weight gain in response to supportive care and tumor control (FIG. 13).

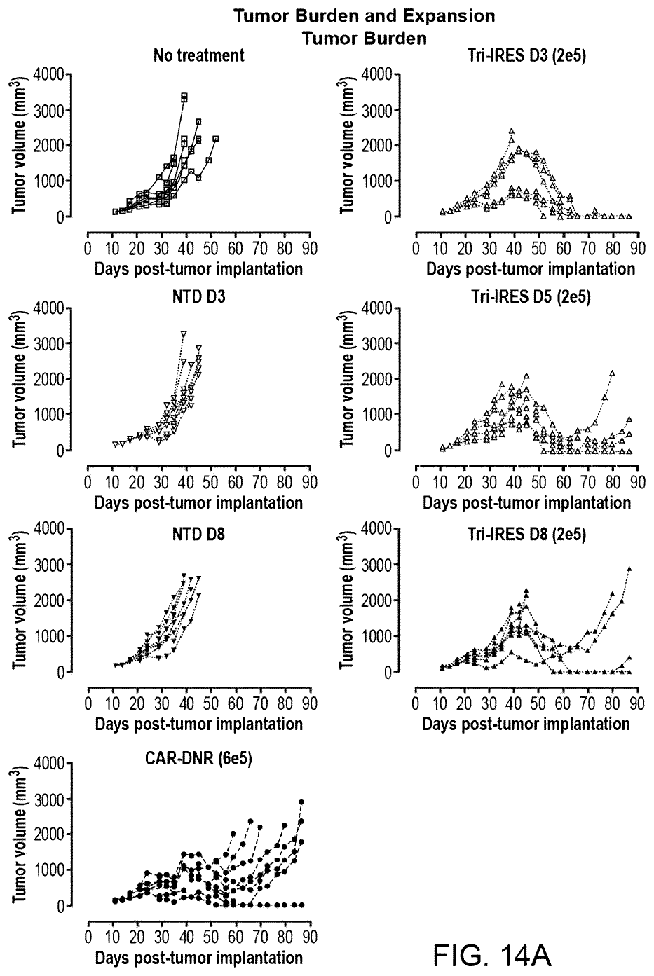

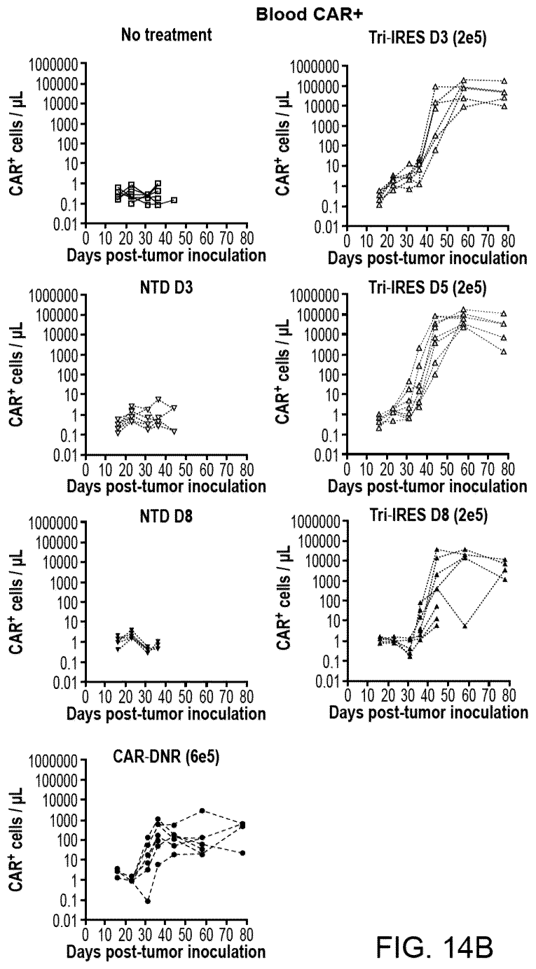

D3, D5 and D8 tricistronic constructs administered at a 2e5 (2×105) CAR dose were more potent than the bicistronic CAR administered at a higher 6e5 CAR dose, based on tumor burden. At the 6e5 CAR dose, 1 of 8 mice showed complete tumor control in the bicistronic CAR group by end of study, while 5, 3, and 1 of 8 mice showed complete tumor control in the tricistronic D3, D5 and D8 groups at a lower 2e5 dose, respectively. At the 2e5 dose, mice treated with D3 or D5 harvested tricistronic CAR+ T cells were more potent than D8 harvested cells. At this dose, tumor control was seen in 5 of 8 mice treated with D8 cells, with 1 surviving to study end with no apparent sign of tumor relapse. In contrast, in mice treated with D3 or D5 harvested cells, tumor control was seen in 6 to 7 of 8 mice of these groups, with 4 to 5 of 8 surviving to study end (FIG. 14A-B).

In the peripheral blood, tricistronic constructs at the lower 2e5 dose had greater CAR+ T-cell expansion and persistence compared to the bicistronic CAR administered at a higher 6e5 dose (FIG. 14A-B).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A polynucleotide, comprising a promoter operatively linked to,
(a) a first coding sequence encoding a chimeric antigen receptor (CAR),
(b) a second coding sequence encoding a TGF-beta dominant negative receptor (TGFβ DNR),
(c) a third coding sequence encoding a membrane-bound IL15 protein (mbIL15),
(L1) a first linker, between (a) and (b), encoding a first self-cleaving peptide, and
(L2) a second linker, between (b) and (c), encoding a second self-cleaving peptide or comprising a ribosome entry site,
wherein the CAR comprises an antigen-binding fragment specific to Glypican 3 (GPC3).

2. The polynucleotide of claim 1, wherein the promoter is an EFla promoter.

3. The polynucleotide of claim 2, wherein the EFla promoter comprises the nucleotide sequence of SEQ ID NO:1.

4. The polynucleotide of claim 1, wherein the first self-cleaving peptide is a 2A self-cleaving peptide.

5. The polynucleotide of claim 4, wherein the 2A self-cleaving peptide is selected from the group consisting of T2A, P2A, and E2A.

6. The polynucleotide of claim 4, wherein the first self-cleaving peptide comprises T2A.

7. The polynucleotide of claim 6, wherein the T2A comprises the amino acid sequence of SEQ ID NO:2.

8. (canceled)

9. The polynucleotide of claim 7, wherein the second linker (L2) comprises an internal ribosome entry site (IRES).

10. The polynucleotide of claim 9, wherein the IRES is selected from the group consisting of Encephalomyocarditis virus (EMCV) IRES, murine Stem Cell Virus (mSCV) IRES, Picornavirus IRES, Aphthovirus IRES, Kaposi's sarcoma-associated herpesvirus IRES, Hepatitis A IRES, Hepatitis C IRES, Pestivirus IRES, Cripavirus IRES, Rhopalosiphum padi virus IRES, and combinations thereof.

11. The polynucleotide of claim 9, wherein the IRES is an EMCV IRES.

12. The polynucleotide of claim 11, wherein the EMCV IRES comprises the nucleotide sequence of SEQ ID NO:5.

13-21. (canceled)

22. The polynucleotide of claim 1, wherein the CAR comprises a single chain fragment (scFv).

23. The polynucleotide of claim 22, wherein the scFv comprises a heavy chain variable region (V_{H}) comprising an amino acid sequence selected from the group consisting of SEQ ID NO: 16 and 23-24 and a light chain variable region (V_{L}) comprising an amino acid sequence selected from the group consisting of SEQ ID NO:18 and 25.

24. The polynucleotide of claim 23, wherein the V_{H} comprises the amino acid sequence of SEQ ID NO:16 and the V_{L} comprises the amino acid sequence of SEQ ID NO:18.

25. The polynucleotide of claim 24, wherein the CAR further comprises a CD3 zeta signaling domain.

26. The polynucleotide of claim 25, wherein the CD3 zeta signaling domain comprises the amino acid sequence of SEQ ID NO:21.

27. The polynucleotide of claim 1, wherein the CAR comprises the amino acid sequence of SEQ ID NO:14.

28. The polynucleotide of claim 1, wherein the TGFβ DNR comprises an extracellular domain (ECD) from a TGF-β receptor, and a transmembrane domain (TMD), and lacks amino acid residues responsible for signaling and phosphorylation present in a wild-type TGF-β receptor.

29. The polynucleotide of claim 28, wherein the ECD is from TGF-βRI or TGF-βRII.

30. The polynucleotide of claim 29, wherein the TGFβ DNR comprises the amino acid sequence of SEQ ID NO:8.

31. The polynucleotide of claim 1, wherein the mbIL15 comprises an IL-15 domain, a first linker linking the IL-15 domain to an IL-15Rα sushi domain, and a transmembrane domain.

32. The polynucleotide of claim 31, wherein the mbIL15 comprises an amino acid sequence selected from the group consisting of SEQ ID NO:9-12.

33. The polynucleotide of claim 1, wherein the (a), (b) and (c) are all downstream from the promoter.

34. The polynucleotide of claim 33, wherein the (a), (b) and (c) are disposed sequentially, proximal to distal, from the promoter.

35. A vector comprising the polynucleotide of claim 1.

36. The vector of claim 35, which is a plasmid or a viral vector.

37-50. (canceled)
